(19) United States
(12) Patent Application Publication
(10) Pub. No.: US 2018/0125752 A1
(43) Pub. Date: May 10, 2018

(54) GLASS BIO-CONTAINER HOLDERS AND ASSEMBLIES

(71) Applicant: Corning Incorporated, Corning, NY (US)

(72) Inventors: Michael Charles Gerrish, Corning, NY (US); Yulong Hong, Painted Post, NY (US); William Joseph Lacey, North Andover, MA (US); David John McEnroe, Corning, NY (US); Aniello Mario Palumbo, Painted Post, NY (US)

(21) Appl. No.: 15/799,160

(22) Filed: Oct. 31, 2017

Related U.S. Application Data
(60) Provisional application No. 62/417,546, filed on Nov. 4, 2016.

(51) Int. Cl. A61J 1/03; C12M 1/00; A61J 1/10; A61J 1/14

(52) U.S. Cl. CPC A61J 1/03; A61J 1/1468; A61J 1/10; C12M 23/02

(57) ABSTRACT

A bio-container assembly that includes a bio-container having an interior surface, an exterior surface, and a container thickness from about 0.2 mm to about 2 mm; at least one port coupled to the bio-container; a holder element comprising a polymeric material; and a cushion affixed to the holder element in contact with one or more primary exterior surfaces of the bio-container. Further, the element and the cushion are configured to enclose the bio-container. The holder element may also include a window, the bio-container being viewable through the window. The holder element may also include one or more of the following features affixed to it: a handle, a plurality of feet, a plurality of ribs, and a plurality of tabs.

### CROSS-REFERENCE TO RELATED APPLICATIONS

The application claims the benefit of priority under 35 U.S.C. § 119 of U.S. Provisional Application Ser. No. 62/417,546 filed on Nov. 4, 2016 the contents of which are relied upon and incorporated herein by reference in their entirety as if fully set forth below.

### BACKGROUND

The present disclosure relates generally to bio-containers, bio-container assemblies and methods of making them and, more particularly, to bio-container holders and assemblies for use in the biopharmaceutical industry, among other industries.

As the biopharmaceutical industry continues to grow, with increased production of parenteral drugs, a trend exists for employing single-use systems in the manufacturing of these products. These single-use systems can reduce overall manufacturing costs as the component costs for these systems are lower than the costs of reusing and turning over multiple-use bio-reactors and associated components, such as stainless steel bio-reactors. A key component in these single-use systems is the container, sometimes referred to as a “Fill & Finish container” or a “bio-container”. These Fill & Finish containers are usually configured to both capture the end product after manufacturing and for transportation of the product to a filling line. These Fill & Finish containers can be fabricated with fairly large volumes, e.g., from 1 to 100 liters; accordingly, each container can hold large amounts of valuable bio-pharmaceutical product and, depending on the particular drug, the value of the product in each container can exceed $1,000,000.

Conventional single-use Fill & Finish containers are typically made from single layers or multi-layers of polymeric and/or elastomeric materials. These polymeric and elastomeric materials include polyethylene terephthalate (“PET”), polyamide (“PA”), ethylene vinyl alcohol (“EVOH”) and ultra-low density polyethylene (“ULDPE') compositions. Some positive attributes of polymeric and elastomeric-based Fill & Finish containers and bio-containers are their relatively low component and manufacturing costs. Another benefit of these materials is that the resulting Fill & Finish container is typically low in weight, which can reduce transportation costs and facilitate ease-of-handling and storage by stacking. In addition, polymeric and elastomeric-based bio-containers can be fabricated in a variety of sizes and shapes.

Nevertheless, single-use Fill & Finish containers and bio-containers fabricated from polymeric and elastomeric materials are subject to some significant drawbacks. One such drawback associated with such bio-containers is the possibility of compounds that may leach from the contact surfaces of the container into, or otherwise react with, the end product. As just one example, some studies have shown that bis (2,4-di-tert-butylphenyl) phosphate (“bDtBPP”), an ingredient in some of these polymeric and elastomeric materials including polyethylene-based materials, can reduce cell culture life and negatively interact with biologic end products within these containers. bDtBPP is just one of the plethora of chemical species extractable from the conventional materials employed in the construction of single-use bioprocess containers. In the future, other extractable chemical species may also be identified as having the effect of reducing cell culture life and negatively interacting with biologic end products within these containers. Another drawback associated with these polymeric and elastomeric materials is their susceptibility to tearing, puncturing or degrading, all of which can lead to unwanted exposure of the end product to air and/or leakage.

Accordingly, there is a need for durable, light-weight Fill & Finish containers, bio-containers and assemblies that have no susceptibility to leaching of container materials and by-products into the contents (e.g., biological, pharmaceutical and bio-pharmaceutical end products, cell cultures, biologics, etc.) within these containers. There is also a need for methods of making such bio-containers and assemblies that are low in cost. Methods of manufacturing these bio-containers preferably allow for high design flexibility with regard to container shape and volume.

### SUMMARY

A first aspect of the disclosure pertains to a holder for a bio-container that includes a holder element comprising a polymeric material and a window; and a cushion affixed to the holder element in contact with one or more primary exterior surfaces of the bio-container. Further, the element, cushion and window are configured to enclose the bio-container, the bio-container viewable through the window.

According to a second aspect, the bio-container of aspect 1 is provided, further comprising a handle affixed to the holder element.

According to a third aspect, the bio-container of aspect 1 or 2 is provided, wherein the cushion is further configured to be in contact with side edges of the bio-container.

According to a fourth aspect, the bio-container of any one of aspects 1-3 is provided, further comprising a plurality of ribs affixed to the holder element that are located and sized such that a side edge of the holder and the bio-container can rest on a substantially flat surface.

According to a fifth aspect, the bio-container of any one of aspects 1-4 is provided, further comprising a plurality of feet affixed to the holder element that are located and sized such that the holder element can be stacked onto another holder element and a primary side of the holder element can rest on a substantially flat surface.

According to a sixth aspect, the bio-container of any one of aspects 1-5 is provided, further comprising a plurality of tabs affixed to the holder element, the tabs configured to secure the holder over the bio-container.

According to a seventh aspect, the bio-container of any one of aspects 1-5 is provided, further comprising a plurality of tabs affixed to the holder element, the tabs configured to removably fasten the holder over the bio-container.

An eighth aspect of the disclosure pertains to a holder for a bio-container that includes an upper and lower holder element, each element comprising a polymeric material; an upper cushion affixed to the upper element in contact with an upper peripheral exterior surface of the bio-container; and a lower cushion affixed to the lower element in contact with a lower peripheral exterior surface of the bio-container. Further, the upper and lower elements are configured to enclose the bio-container.

According to a ninth aspect, the bio-container of aspect 8 is provided, further comprising a handle affixed to at least one of the holder elements.

According to a tenth aspect, the bio-container of aspect 8 or 9 is provided, wherein the upper and lower cushions are further configured to be in contact with side edges of the bio-container.

According to an eleventh aspect, the bio-container of any one of aspects 8-10 is provided, further comprising a plurality of ribs affixed to at least one of the holder elements that are located and sized such that a side edge of the holder and the bio-container can rest on a substantially flat surface.

According to a twelfth aspect, the bio-container of any one of aspects 8-11 is provided, further comprising a plurality of feet affixed the lower holder element that are located and sized such that the holder element can be stacked onto another holder element and a primary side of the holder element can rest on a substantially flat surface.

According to a thirteenth aspect, the bio-container of any one of aspects 8-12 is provided, further comprising a plurality of tabs affixed to at least one of the holder elements, the tabs configured to secure the holder over the bio-container.

According to a fourteenth aspect, the bio-container of any one of aspects 8-12 is provided, further comprising a plurality of tabs affixed to at least one of the holder elements, the tabs configured to removably fasten the holder over the bio-container.

A fifteenth aspect of the disclosure pertains to a bio-container assembly that includes a bio-container having an interior surface, an exterior surface, and a container thickness from about 0.2 mm to about 2 mm; at least one port coupled to the bio-container; a holder element comprising a polymeric material; and a cushion affixed to the holder element in contact with one or more primary exterior surfaces of the bio-container. Further, the element and the cushion are configured to enclose the bio-container. In addition, the bio-container has a glass composition comprising no materials that are leachable in excess of a Permitted Daily Exposure (PDE) upon exposure to contents of the bio-container.

According to a sixteenth aspect, the bio-container assembly of aspect 15 is provided, wherein the bio-container further comprises a glass layer spanning the container thickness.

According to a seventeenth aspect, the bio-container assembly of aspect 15 or 16 is provided, wherein the bio-container comprises a compressive stress region that extends to a selected depth in the container thickness and a maximum compressive stress at one or both of the interior and exterior surfaces.

According to an eighteenth aspect, the bio-container assembly of aspect 17 is provided, wherein the compressive stress region comprises a plurality of ion-exchangeable ions and a plurality of ion-exchanged ions.

According to a nineteenth aspect, the bio-container assembly of any one of aspects 15-18 is provided, wherein the container further comprises a laminated sheet having a plurality of glass layers spanning the container thickness.

According to a twentieth aspect, the bio-container assembly of aspect 19 is provided, wherein the plurality of glass layers comprise glass compositions with a coefficient of thermal expansion (CTE) mismatch and the compressive stress region is based at least in part on the CTE mismatch.

According to a twenty-first aspect, the bio-container assembly of aspect 19 is provided, wherein the plurality of glass layers comprise a core layer, outer clad layer and an inner clad layer, the clad layers having a lower coefficient of thermal expansion (CTE) than the core layer and each of the layers having a softening point within 200° C. of the softening point of the other layers.

According to a twenty-second aspect, the bio-container assembly of any one of aspects 15-21 is provided, wherein the bio-container comprises a first half, a second half, and a seam that joins the halves.

According to a twenty-third aspect, the bio-container assembly of any one of aspects 15-22 is provided, wherein the bio-container further comprises an interior volume from about 1 L to about 200 L.

Additional features and advantages will be set forth in the detailed description which follows, and will be readily apparent to those skilled in the art from that description or recognized by practicing the embodiments as described herein, including the detailed description which follows, the claims, as well as the appended drawings.

It is to be understood that both the foregoing general description and the following detailed description are merely exemplary, and are intended to provide an overview or framework to understanding the nature and character of the disclosure as it is claimed.

The accompanying drawings are included to provide a further understanding of principles of the disclosure, and are incorporated in, and constitute a part of, this specification. The drawings illustrate one or more embodiment(s) and, together with the description, serve to explain, by way of example, principles and operation of the disclosure. It is to be understood that various features of the embodiments disclosed in this specification and in the drawings can be used in any and all combinations. By way of non-limiting examples, the various features of the disclosure may be combined with one another according to the following aspects.

### BRIEF DESCRIPTION OF THE DRAWINGS

These and other features, aspects and advantages of the present disclosure are better understood when the following detailed description of the disclosure is read with reference to the accompanying drawings, in which:

FIG. 1 is a schematic, perspective view of a bio-container with a plurality of ports according to an aspect of the disclosure.

FIG. 1A is a cross-sectional view of the bio-container depicted in FIG. 1 along line IA-IA.

FIG. 1B is an enlarged view of a portion of the bio-container depicted in FIG. 1, as configured with a plurality of glass layers spanning the thickness of the bio-container according to an aspect of the disclosure.

FIG. 1C is an enlarged view of a portion of the bio-container depicted in FIG. 1, as configured with a plurality of glass layers spanning the thickness of the bio-container and comprising an inner clad, a core and an outer clad layer according to an aspect of the disclosure.

FIG. 1D is an enlarged view of a portion of the bio-container depicted in FIG. 1, as configured with a compressive stress region that extends to a selected depth from the interior and exterior surfaces of the bio-container according to an aspect of the disclosure.

FIG. 2 is a schematic, perspective view of a bio-container with a plurality of ports, a first half, a second half and a seam that joins the halves according to an aspect of the disclosure.

FIG. 2A is a cross-sectional view of the bio-container depicted in FIG. 2 along line IIA-IIA.

FIG. 3 is a top-down, plan view of a mold having a mold surface comprising a plurality of vacuum holes for fabricating a glass sheet into a bio-container half according to an aspect of the disclosure.

FIG. 3A is a schematic, perspective view of a glass sheet positioned on the mold depicted in FIG. 3 during the heating and de-pressurizing steps of a method of making a bio-container half according to an aspect of the disclosure.

FIG. 3B is a schematic, perspective view of a first bio-container half fabricated from a mold as depicted in FIG. 3 according to an aspect of the disclosure.

FIG. 3C is a schematic, perspective view of a second bio-container half fabricated from a mold as depicted in FIG. 3 according to an aspect of the disclosure.

FIG. 3D is a schematic, perspective view of a bio-container fabricated from two bio-container halves, as depicted in FIGS. 3B and 3C, according to an aspect of the disclosure.

FIG. 4A is a schematic, perspective view of a bio-container half that was fabricated within a mold, such as depicted in FIG. 3, after the heating and de-pressurizing steps of a method of making a bio-container half, according to an aspect of the disclosure.

FIG. 4B is a schematic, perspective view of a bio-container half, as depicted in FIG. 4A, being subjected to a pressing operation with a die to further form the ports of the bio-container half according to an aspect of the disclosure.

FIG. 4C is a schematic, perspective view of a bio-container half, as depicted in FIG. 4A, being subjected to a pressing operation with a die and positional frame to further form the ports of the bio-container half according to an aspect of the disclosure.

FIG. 5 is a top-down, plan view of a shaped glass sheet configured for forming a bio-container half according to an aspect of the disclosure.

FIG. 6 is a schematic, perspective view of a bio-container half in a mold, such as depicted in FIG. 3, being subjected to a trimming process with a trim assembly according to an aspect of the disclosure.

FIG. 6A is a schematic, perspective view of a trim assembly with an electrical lead configuration that can be employed in the trimming process to trim material around the circumference and in the port regions of the bio-container half depicted in FIG. 6 according to an aspect of the disclosure.

FIG. 6B is a schematic, perspective view of a trim assembly with an electrical lead configuration that can be employed in the trimming process to trim material around the circumference and between the port regions of the bio-container half depicted in FIG. 6 according to an aspect of the disclosure.

FIG. 6C is a schematic, perspective view of a trim assembly with an electrical lead configuration that can be employed in the trimming process to trim material around the circumference and between the port regions of the bio-container half depicted in FIG. 6 according to an aspect of the disclosure.

FIG. 7 is a schematic, perspective view of two bio-container halves and tubes installed in their port regions, all contained within a sealing assembly comprising electrical lead configurations for joining the halves with a direct sealing process according to an aspect of the disclosure.

FIG. 7A is a schematic, top-down perspective view of a bio-container, as sealed with the sealing assembly depicted in FIG. 7, according to an aspect of the disclosure.

FIG. 7B is a schematic, side perspective view of the bio-container depicted in FIG. 7A.

FIG. 8A is a schematic, top-down perspective view of a bio-container half with a frit applied to sealing and port regions of the half according to an aspect of the disclosure.

FIG. 8B is a schematic, top-down perspective view showing a frit applied to a sealing region and tubes installed in the port regions of a bio-container half according to an aspect of the disclosure.

FIG. 8C is a schematic, perspective view of two bio-container halves, such as depicted in FIGS. 8A & 8B, within a single-block sealing assembly containing an electrical lead configuration for joining the halves with frit according to an aspect of the disclosure.

FIG. 8D is a schematic, perspective view of two bio-container halves, such as depicted in FIGS. 8A & 8B, within a dual-block sealing assembly containing electrical lead configurations for joining the halves with frit according to an aspect of the disclosure.

FIG. 8E is a schematic, top-down perspective view of a bio-container, as sealed with a sealing assembly such as depicted in FIGS. 8C or 8D, according to an aspect of the disclosure.

FIG. 8F is a schematic, side perspective view of the bio-container depicted in FIG. 8E.

FIG. 9A is a schematic, exploded perspective view of a bio-container assembly that includes a bio-container and a holder element according to an aspect of the disclosure.

FIG. 9B is a schematic, exploded side perspective view of the bio-container assembly depicted in FIG. 9A.

FIG. 9C is a schematic, top-down perspective view of the bio-container assembly depicted in FIG. 9A.

FIG. 9D is a schematic perspective view of the bio-container assembly depicted in FIG. 9A.

FIG. 10A is a schematic, top-down perspective view of a bio-container assembly that includes a bio-container, a holder element and a translucent panel according to another aspect of the disclosure.

FIG. 10B is a schematic, exploded perspective view of the bio-container assembly depicted in FIG. 10A showing the panel disassembled from the assembly.

FIG. 10C is a cross-sectional view of the bio-container assembly depicted in FIG. 10A.

### DETAILED DESCRIPTION

In the following detailed description, for purposes of explanation and not limitation, example embodiments disclosing specific details are set forth to provide a thorough understanding of various principles of the present disclosure. However, it will be apparent to one having ordinary skill in the art, having had the benefit of the present disclosure, that the present disclosure may be practiced in other embodiments that depart from the specific details disclosed herein. Moreover, descriptions of well-known devices, methods and materials may be omitted so as not to obscure the description of various principles of the present disclosure. Finally, wherever applicable, like reference numerals refer to like elements.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

Directional terms as used herein—for example up, down, right, left, front, back, top, bottom—are made only with reference to the figures as drawn and are not intended to imply absolute orientation.

Unless otherwise expressly stated, it is in no way intended that any method set forth herein be construed as requiring that its steps be performed in a specific order. Accordingly, where a method claim does not actually recite an order to be followed by its steps, or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

As used herein, the singular forms “a,” “an” and “the” include plural referents unless the context clearly dictates otherwise. Thus, for example, reference to a “component” includes aspects having two or more such components, unless the context clearly indicates otherwise.

Aspects of the disclosure generally pertain to bio-containers, bio-container assemblies and methods of making the same. The bio-containers can include a single-use container having a glass composition with no propensity for leaching of organic and inorganic materials upon exposure to contents within the bio-containers including, but not limited to, biologics and pharmaceutical end products. Further, the bio-containers can include one or more ports for introducing and dispensing such end products. The bio-container assemblies of the disclosure can employ a polymeric holder element to house the bio-container, facilitate storage and handling, and increase durability. The methods of making these bio-containers can involve vacuum molding steps for fabricating glass sheets into portions or halves of the final, single-use container. These methods may also include high-temperature steps for sealing these portions and halves together into the final, single-use container.

The bio-container design and processing technologies of the disclosure offer several advantages. A primary advantage of these bio-containers, particularly over conventional bio-containers made from polymeric and elastomeric materials, is their resistance to material and by-product leaching into the contents (e.g., biological, pharmaceutical and bio-pharmaceutical end products, cell cultures, biologics, etc.) housed in the container. A similar benefit of the bio-containers of the disclosure is improved resistance to moisture, oxygen, carbon dioxide and other environmental contaminants through reduced diffusivity of these substances through the thickness of the glass container. Another advantage of these bio-containers is their high durability. As the single-use container employed in these bio-containers is fabricated from one or more glass sheets, the container will have high resistance to tearing, puncturing, and other permanent deformation. Further, the resistance of these bio-containers to container material and by-product leaching into their contents add to the overall durability of the container as its composition will not significantly change during its application lifetime. Another advantage of the bio-containers (and associated processing technologies) of the disclosure is that they can be configured with the same or similar shape and volume versatility as conventional bio-containers fabricated from polymeric and elastomeric materials. Still further, the bio-containers of the disclosure are light-weight, and similarly versatile with regard to shipping and handling as compared to conventional bio-containers fabricated from polymeric and elastomeric materials. An addition advantage offered by the bio-containers and processing technologies of the disclosure is the capability of some embodiments to include antimicrobial exterior surfaces, which can increase the versatility of these containers to be used in various settings, including those with high-traffic.

These bio-container and bio-container assembly technologies also benefit from the polymeric holder technologies of the disclosure. In particular, the holders can be configured to enclose the bio-containers, adding to the overall durability of the system, including from shock and impact evolutions during use, shipment and handling. Another benefit of the holder technologies of the disclosure is that the holders can make the bio-containers easier to use, store and transport. For example, the typical rigidity of these holders allows them to be designed in various configurations suitable for enclosing the bio-container while simplifying stacking and storage. Another benefit of these holder technologies is that they can be readily labeled, stamped, coded, etc. for application-related uses, storage and handling without risk to the bio-container and the product. A further benefit of the holder technologies is that they can reduce the contamination risks associated with the bio-containers and product by serving as a barrier (e.g., with handles, tabs, etc.) to displace manual contacts away from the bio-containers and product.

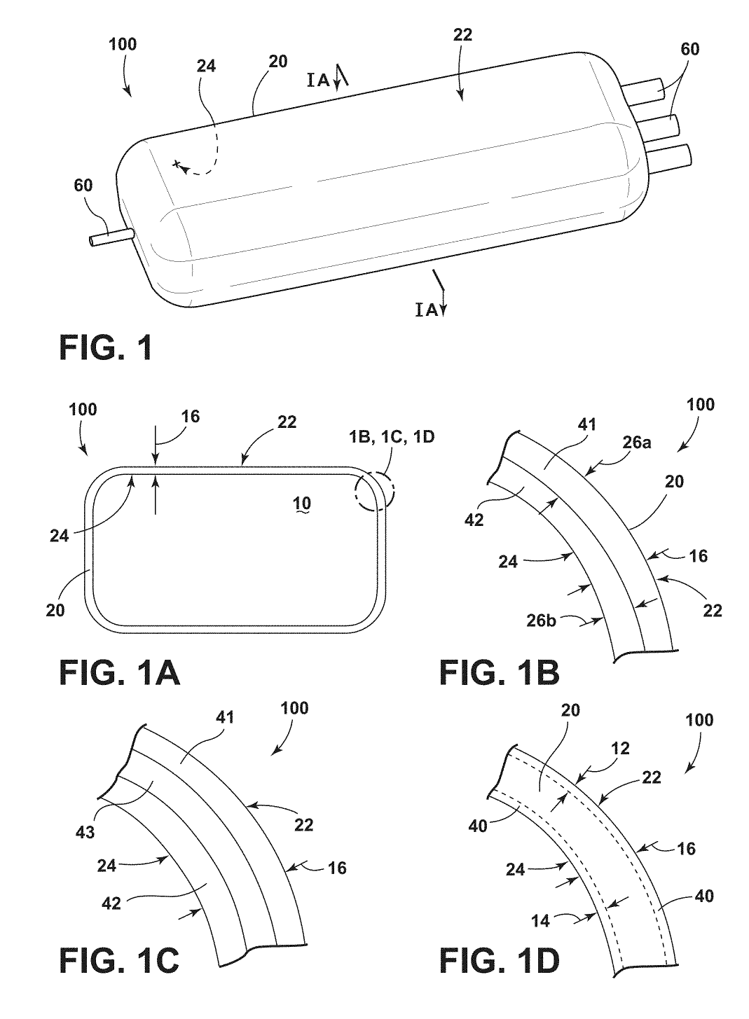

Referring to FIGS. 1 and 1A, a bio-container 100 according to an aspect of the disclosure is depicted. The bio-container 100 includes a single-use container 20 having an exterior surface 22, an interior surface 24, and a container thickness 16. Further, the bio-container 100 includes at least one tube 60, as coupled to the single-use container 20. Together, the tube or tubes 60 and the interior surface 24 serve to define an interior 10 of the single-use container 20 of the bio-container 100.

In addition, the single-use container 20 can be configured with a glass composition which includes no materials (e.g., As, Cd, Hg, Pb, Co, Mo, Se, V, Ag, Au, Ir, Os, Pd, Pt, Rh, Ru, Tl, Ba, Cr, Cu, Li, Ni, Sb and Sn) that are leachable in excess of a Permitted Daily Exposure (PDE) upon exposure to contents of the container (e.g., biological, pharmaceutical and bio-pharmaceutical end products, cell cultures, biologics, etc.). That is, the glass composition of the container 20 can be selected such that the constituents of the container 20 do not readily leach into the contents stored in the interior 10 10 at levels above the PDE, as set forth in the “Guideline for Elemental Impurities,” Draft Consensus Guideline of the International Conference on Harmonisation of Technical Requirements for Registration of Pharmaceuticals for Human Use, Jul. 26, 2013, incorporated by reference in its entirety. In certain aspects, the glass composition for the single-use container 20 is selected to ensure leach resistance in view of particular, classes or groups of contents (e.g., biological, pharmaceutical and bio-pharmaceutical end products, cell cultures, biologics, etc.) targeted for housing within the bio-container 100 that employs the container 20. Preferably, the container 20 is constructed such that the contents in the interior 10 is in contact with a glass composition that is selected to ensure leach resistance; consequently, other portions of the container 20 not directly in contact with the contents can, in some implementations, have a composition that does not possess the same level of leach resistance as the portion of the container 20 in contact with the contents.

Suitable glass compositions for the single-use container 20 include Corning® Inc. Gorilla®, Valor®, Eagle XG® and Pyrex® glass compositions. Further, such glass compositions can be found in U.S. Pat. No. 7,524,784 and U.S. Patent Application Publication Nos. 2014/0120279, 2013/0216742, 2013/0202823, and 2013/0196094, the salient portions of which related to glass compositions are hereby incorporated by reference in this disclosure.

Among other uses, the bio-container 100 can function to store, transport and dispense contents (e.g., biological, pharmaceutical and bio-pharmaceutical end products, cell cultures, biologics, etc.) contained within the interior 10 of the single-use container 20. The one or more tubes 60 (and/or ports 30) coupled to the container 20 allow a user to transfer these contents into and out of the container 20. In certain implementations, the bio-container 100 can serve as a reaction and/or cell culturing vessel for fabricating these contents. Accordingly, precursors, catalysts, nutrients, and other constituents of the contents, or necessary for the fabrication of such contents, can be introduced into the container 20 via the port or tubes 60. The bio-container 100 can then be subjected to additional processing steps (e.g., heating, agitation, introduction of certain gaseous environments and/or pressure through the tubes 60, etc.) to produce the desired contents from the precursors and other constituents introduced into the container 20. Accordingly, the bio-container 100 can function as a bio-reactor in some implementations.

Referring again to FIGS. 1 and 1A, the bio-container 100 is depicted with a rectangular-shaped, single use container 20. Other implementations of the bio-container 100 may employ other shapes for the container 20, including but not limited to cubic, spherical and cylindrical shapes. In addition, some embodiments of the bio-container 100 can be configured with single-use containers 20 having one or more pleats or pleated regions (not shown) to facilitate storage of the bio-container in a two-dimensional configuration, e.g., prior to introduction of an end product or other contents via the tubes 60 into the interior 10. Certain embodiments of the bio-container 100 with such pleats or pleated regions can employ a container 20 with a glass composition, one or more compressive stress regions (i.e., as described later in the disclosure), and a relatively low container thickness 16 to facilitate folding of the pleats or pleated regions. For example, such containers 20 can be configured with a container thickness 16 that ranges from about 0.1 mm to about 0.5 mm, thus ensuring some versatility of the single-use container 20 having a glass composition.

Again referring to FIGS. 1 and 1A, the single-use container 20 of the bio-container 100 can be configured with a relatively large range of container thicknesses 16. In certain implementations, the container thickness 16 can range from about 0.05 mm to about 3 mm, preferably between 0.1 mm and 2 mm. For example, the container thickness 16 can be about 0.1 mm, 0.11 mm, 0.12 mm, 0.13 mm, 0.14 mm, 0.15 mm, 0.16 mm, 0.17 mm, 0.18 mm, 0.19 mm, 0.2 mm, 0.3 mm, 0.4 mm, 0.5 mm, 0.6 mm, 0.7 mm, 0.8 mm, 0.9 mm, 1 mm, 1.1 mm, 1.2 mm, 1.3 mm, 1.4 mm, 1.5 mm, 1.6 mm, 1.7 mm, 1.8 mm, 1.9 mm, 2 mm, 2.1 mm, 2.2 mm, 2.3 mm, 2.4 mm, 2.5 mm, 2.6 mm, 2.7 mm, 2.8 mm, 2.9 mm, 3 mm and other thickness between these exemplary thicknesses.

As depicted in FIGS. 1 and 1A, the bio-container 100 can employ a single-use container 20 sized to accommodate end product volumes and volumes of other contents within the interior 10 that range from about 0.5 L to about 200 L. For example, the interior 10 of the single-use container 20 can be 0.5 L, 1 L, 2 L, 3 L, 4 L, 5 L, 10 L, 20 L, 25 L, 30 L, 40 L, 50 L, 60 L, 70 L, 75 L, 80 L, 90 L and 100 L. In certain implementations of the bio-container 100, the single-use container 20 can be configured with one or more dividing regions (not shown) within the interior 10 to divide the available volume into two or more regions to accommodate multiple types of contents (e.g., biological, pharmaceutical and bio-pharmaceutical end products, cell cultures, biologics, etc.) within the same bio-container 100. In such configurations, the multiple tubes 60 are coupled to particular locations of the container 20 such that at least one tube 60 is available to transfer and dispense the contents into and out of each of the regions of the container.

As also depicted in exemplary fashion in FIGS. 1 and 1A, the bio-container 100 can employ a single-use container 20 with a single glass layer that spans the container thickness 16 in certain embodiments. Other configurations of the single-use container 20 can employ two or more glass layers (e.g., as a laminated sheet) that span the container thickness 16. These multiple glass layers may have the same glass composition in some embodiments; in other embodiments, one or more of the glass layers can be configured with a glass composition that differs from the compositions of the other layers employed in the container 20 across the container thickness 16. For example, FIG. 1B depicts a configuration of the bio-container 100 with a single-use container 20 having an outer clad layer 41 and an inner clad layer 42 that span the container thickness 16. As another example, FIG. 1C depicts a configuration of the bio-container 100 with a single-use container 20 having an outer clad layer 41, inner clad layer 42 and core layer 43 (i.e., as situated between the clad layers 41, 42) that span the container thickness 16. In addition, each of the glass layers within the single-use container 20 that span the container thickness 16 may have roughly the same thickness or, in other implementations, may possess differing thicknesses. In an exemplary implementation of the bio-container 100 with a single-use container 20 as depicted in FIG. 1C, the clad layers 41, 42 each have a thickness of about 0.175 mm and the core layer 43 has a thickness of about 0.350 mm; consequently, the container thickness 16 is about 0.700 mm in this configuration of the bio-container 100.

As shown in FIG. 1D, certain embodiments of the bio-container 100 employ a single-use container 20 with a compressive stress region 40 that extends to a selected depth 12, 14 within the container thickness 16. In the embodiment shown in FIG. 1D, the compressive stress region 40 is present at both of the exterior and interior surfaces 22, 24 of the container 20, and extends to selected depths 12, 14. The compressive region 40 that extends to the selected depth 12 can have the same or a differing stress distribution as compared to the compressive stress region 40 that extends to the selected depth 14. More generally, the compressive stress associated with the compressive stress region 40 ensures that any cracks, defects and/or flaws that develop in the single-use container 20, particularly at the exterior and interior surfaces 22, 24 do not continue to propagate within the container 20 leading to a failure. Ultimately, as the strength of glass articles is dependent on the flaw distribution within the part, the addition of a compressive stress region 40 in the single-use container 20 tends to increase the strength of the container.

In preferred implementations, the maximum compressive stress associated with the compressive stress region 40 is developed at or in close proximity to the exterior and interior surfaces 22, 24. Further, the selected depths 12, 14 can be roughly equal to or substantially differ from one another. These variations and similarities in the compressive stress region 40 of the single-use container 20, as will be outlined later in the disclosure, can be driven by processing and/or the configuration of one or more glass layers within the container thickness 16. In addition, other implementations of the bio-container 100 can employ a single-use container 20 with a compressive stress region 40 that is present at only the exterior surface 22 or the interior surface 24.

In another implementation, the bio-container 100, as depicted in exemplary form in FIGS. 1 and 1D, can employ a container 20 having a glass composition conducive to the development of a compressive stress region 40 that comprises a plurality of ion-exchangeable ions and a plurality of ion-exchanged ions. For example, Corning® Inc. Gorilla® glass compositions may be employed in such configurations of the container 20. More generally, various ion-exchange processes can be employed to generate the compressive stress region 40 by introducing larger ions (e.g., ion-exchanged ions such as K⁺) into the surface of the glass to replace smaller ions (e.g., ion-exchangeable ions such as Na⁺). For example, a glass sheet can be submerged into a salt bath containing ion-exchanged ions (e.g., KNO₃) at a particular temperature for a predetermined time to ensure that the ion-exchanged ions (K⁺ ions) replace ion-exchangeable ions (e.g., Na⁺ ions) in the glass sheet to a selected depth 12, 14 (also referred to as a “depth-of-layer”). Depending on the ion exchange process conditions employed to develop the compressive stress region 40 within the glass sheet or sheets of the single-use container 20, the compressive stress region 40 can be developed inward from one or more of the exterior and interior surfaces 22, 24 to the selected depths 12, 14. Without being bound by theory, the compressive stress at the surfaces 22, 24 can improve the strength and puncture resistance of the single-use container 20.

According to another embodiment, the bio-container 100, as depicted in exemplary form in FIGS. 1, 1B and 1C, can employ a container 20 configured such that its glass layers (e.g., outer clad layer 41 and inner clad layer 42 as shown in FIG. 1B; outer clad layer 41, core 43, and inner clad layer 42 as shown in FIG. 1C) comprise glass compositions with a coefficient of thermal expansion (“CTE”) mismatch, and the compressive stress region 40 (see FIG. 1D) is based at least in part on the CTE mismatch. In such implementations, an exemplary clad composition can include: about 55% to about 65% SiO₂, about 13% to about 20% Al₂O₃, 0% to about 18% B₂O₃, about 4% to about 12% Na₂O, about 2% to about 12% MgO, 0% to about 10% CaO, and 0% to about 1% SnO₂ (by weight); and an exemplary core composition can include: about 68% to about 78% SiO₂, about 8% to about 15% Al₂O₃, 0% to about 10% B₂O₃, about 10% to about 18% Na₂O, 0% to about 10% K₂O, about 1% to about 10% MgO, 0% to about 10% CaO, and 0% to about 1% SnO₂ (by weight). More generally, as the laminated sheet containing the layers 41, 42 and 43 is cooled after processing into a desired shape (e.g., that of a single-use container 20, container half or the like), the CTE mismatch between the layers results in the development of the compressive stress region 40. In one implementation, the outer and inner clad layers 41, 42 experience a smaller dimensional reduction as compared to the core layer 43 during cooling. In particular, the core layer 43 has a higher CTE than the clad layers 41, 42, resulting in more contraction of the core layer 43 compared to the clad layers 41, 42. Accordingly, the core layer 43 is placed in tension and the clad layers 41, 42 are placed in compression, thus developing the compressive stress region 40. That is, the difference in CTE mismatch reflects the difference in CTEs between the layers employed in the container 20 that span the container thickness 16. In general, a CTE mismatch sufficient to generate a compressive stress region 40 (see FIG. 1D) can be obtained when one or more outer layers (e.g., outer and inner clad layers 41, 42 as shown in FIG. 1C) of the container 20 have a lower CTE than the innermost layer or layers (e.g., the core layer 43).

In one embodiment, the bio-container 100 can employ a single-use container 20 that is configured as shown in FIG. 1C to produce a CTE mismatch-related compressive stress region 40 (see FIG. 1D). In particular, the single-use container 20 includes an outer clad layer 41, inner clad layer 42 and core layer 43 (i.e., as situated between the clad layers 41, 42) that span the container thickness 16. Further, the clad layers 41, 42 comprise respective glass compositions with a lower CTE as compared to the CTE of the glass composition of the core layer 43. Further, the single-use container 20 is configured such that each of the layers 41, 42 and 43 have a softening point within 200° C. of the other layers. Accordingly, this configuration of the single-use container 20 comprises a compressive stress region 40 (see FIG. 1D) that extends to selected depths 12, 14 in the container thickness 16 with a maximum compressive stress at the exterior and interior surfaces 22, 24 at the outer edges of the outer and inner clad layers 41, 42.

According to another embodiment, the bio-container 100 can employ a single-use container 20 that is configured as shown in FIG. 1D to produce an antimicrobial region (not shown) that is coincident with, or overlaps, the compressive stress region 40. Accordingly, the antimicrobial region can extend to selected depths 12, 14 as the compressive stress region 40 or may have different selected depths (not shown), depending on the processing conditions employed to generate the antimicrobial region. In one implementation, such an antimicrobial region is located coincident with or in proximity to a compressive stress region 40 that extends from the exterior surface 22 to a selected depth, e.g., in proximity to selected depth 12. In another implementation, such an antimicrobial region extends from the exterior surface 22 to a selected depth within an outer clad layer 41 of a bio-container with multiple glass layers, e.g., the bio-container depicted in FIG. 1C. According to one embodiment, the antimicrobial region of the single-use container 20 comprises a plurality of ion-exchangeable ions (such as Na⁺) and a plurality of silver ions (e.g., Ag⁺ ions) that have been exchanged with the ion-exchangeable ions. In one implementation, the glass sheet or layers making up the container 20 is immersed into a molten bath containing silver ions at a particular temperature for a prescribed time to develop the antimicrobial region to a selected depth within the container thickness 16.

According to another embodiment, the bio-container 100 can employ a single-use container 20 that is configured as shown in FIG. 1B with two layers. In particular, the outer clad layer 41 can be fabricated with an antimicrobial region and the inner clad layer 42 can be fabricated without any such antimicrobial region. Further, in some aspects, the composition of the outer clad layer 41 can be selected such that it is more susceptible to ion exchanging with silver ions relative to the inner clad layer 42 to facilitate a processing condition in which both layers are immersed into a bath with silver ions, for example. In addition, the composition of the inner clad layer 41 can be selected to facilitate the development of a compressive stress region 40 (see FIG. 1D) through ion exchange processing or otherwise fabricated with higher inherent strength levels.

Without being bound by theory, the foregoing embodiments of bio-container 100 can be combined in various implementations consistent with the principles of the disclosure and in view of application-specific requirements and needs. For example, the bio-container 100 can employ a single-use container 20 fabricated from a laminated glass sheet comprising layers with differing compositions and stress states (e.g., as shown in FIGS. 1, 1C and 1D). In particular, the inner clad layer 42 can be fabricated with a glass composition which includes substantially no organic and inorganic materials that are leachable upon exposure to biologics, pharmaceuticals, bio-pharmaceuticals contained within the interior 10 of the container 20, e.g., a glass composition with no materials that are leachable in excess of a Permitted Daily Exposure (PDE) upon exposure to contents within the container 20. On the other hand, the outer clad layer 41 can be fabricated with a glass composition that is better suited to the development of a compressive stress region 40 to resist scratches, puncturing, tearing and the development of flaws, respectively, all of which can undermine the integrity of the single-use container 20 employed in such a bio-container 100. Similarly, the outer clad layer 41 can be fabricated with a glass composition that is well-suited to the development of an antimicrobial region.

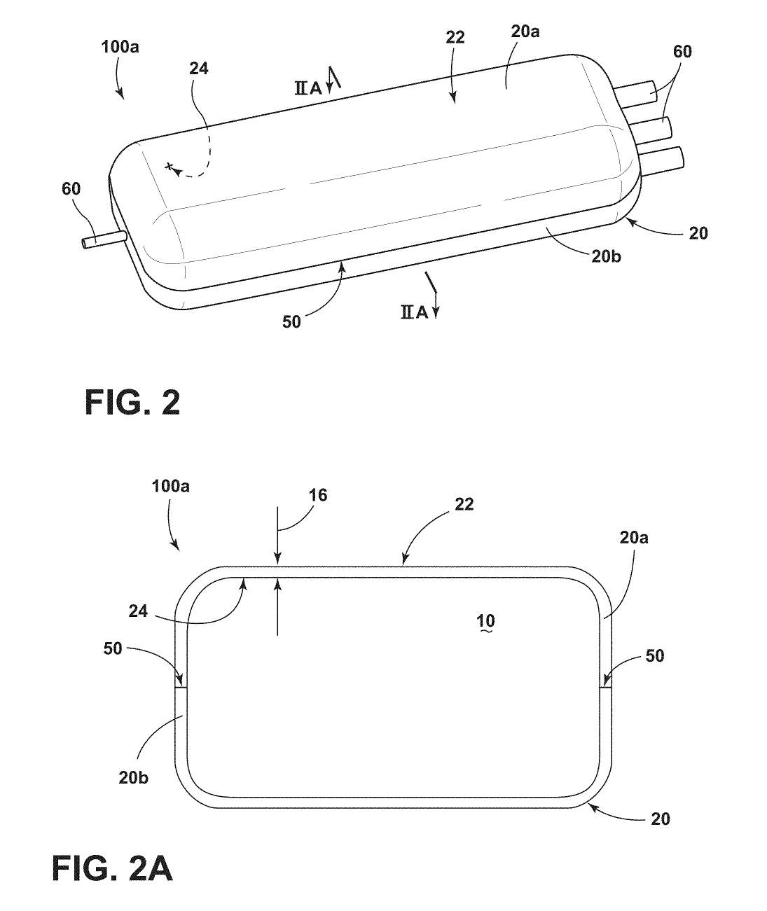

Referring to FIGS. 2 and 2A, a bio-container 100a according to a further aspect of the disclosure is depicted. The bio-container 100a is similar in overall shape and function in comparison to the bio-container 100 (see FIGS. 1 and 1A), and like-numbered elements have the same or similar structure and function. The bio-container 100a includes a single-use container 20 having an exterior surface 22, an interior surface 24, and a container thickness 16. Further, the container includes a first half 20a, a second half 20b and a seam 50 that joins the halves 20a, 20b. In addition, the bio-container 100a includes at least one tube 60, as coupled to the single-use container 20. Together, the tube or tubes 60 and the interior surface 24 of the halves 20a, 20b serve to define an interior 10 of the single-use container 20 of the bio-container 100.

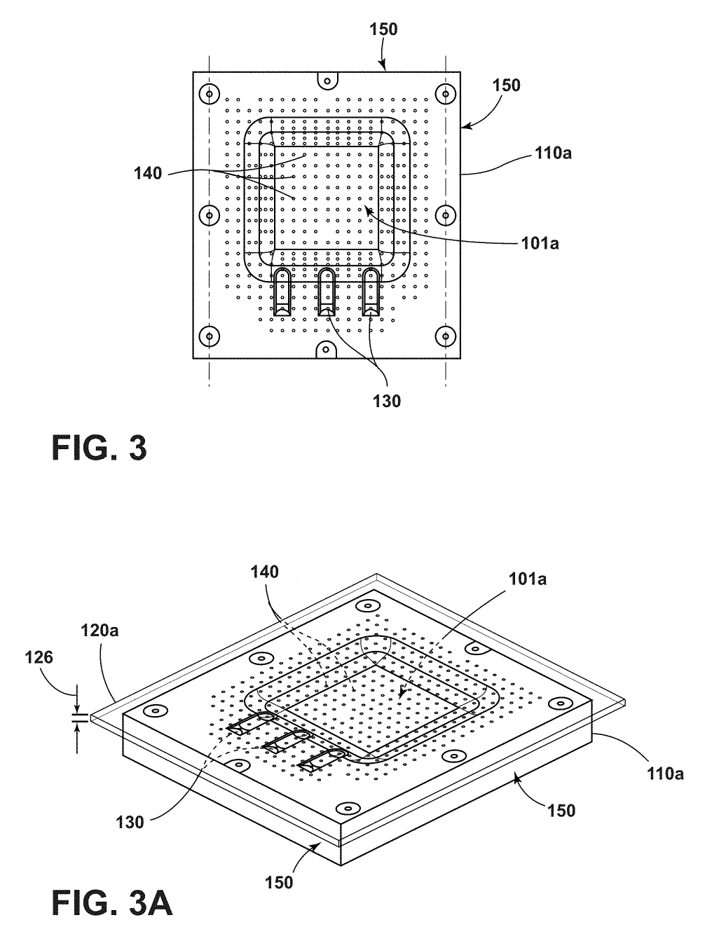

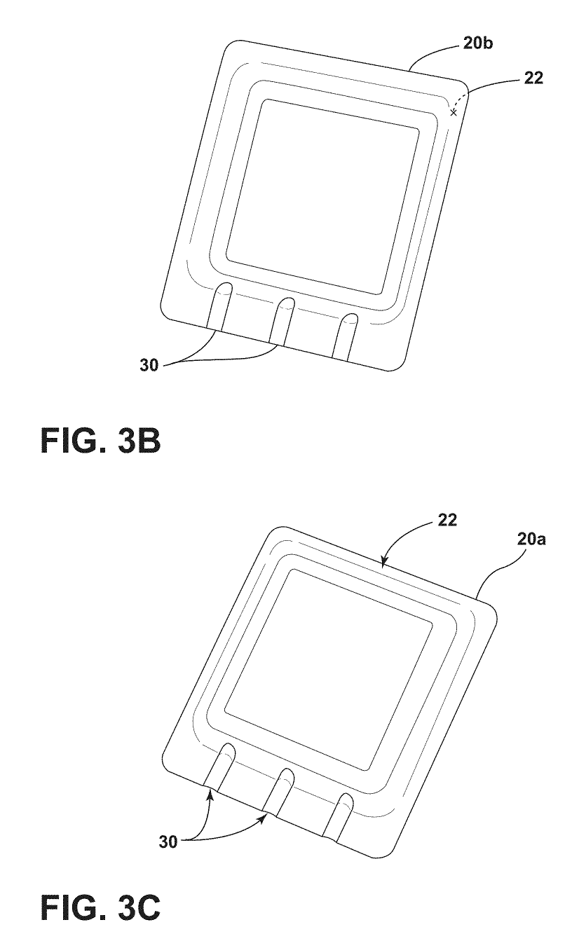

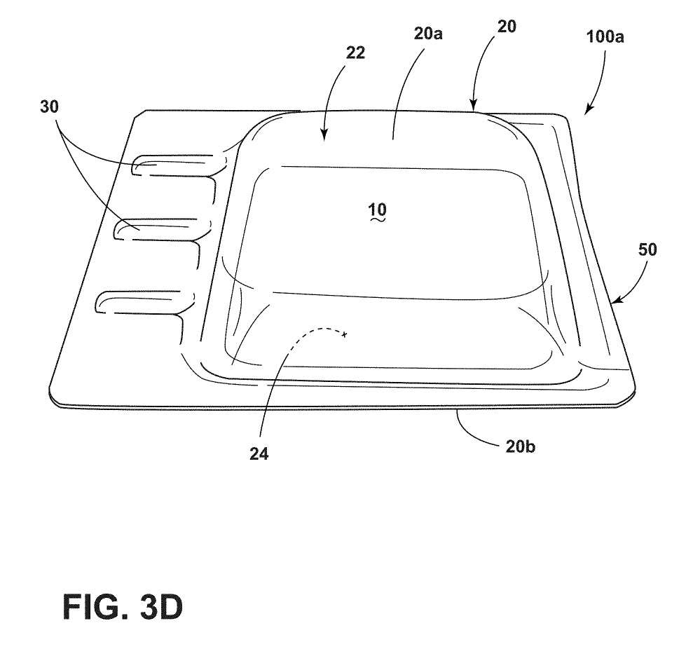

According to another aspect of the disclosure, a method of making a bio-container (e.g., the bio-container 100, 100a detailed in the foregoing disclosure) is provided and schematically depicted in FIGS. 3-3D. The method includes a step of positioning a glass sheet 120a having a thickness 126 on a mold 110a. In some implementations, the glass sheet 120a may have primary surfaces having a surface area of about 0.1 m² to about 3 m². The mold 110a is defined by edges 150, a mold surface 101a, a plurality of vacuum holes 140 and a base (not shown). In addition, the mold 110a includes a port mold surface 130, for forming ports (e.g., the ports 30 of the bio-container 100a as depicted in FIG. 3D) from about 1 to 20 mm in diameter and about 10 to 100 mm in length. In some aspects, a vacuum pump is attached to the vacuum holes 140, which is capable of reducing pressure to between about 10 and about 500 mbar (about 0.010 to 0.5 atm). As shown in FIG. 3A, the glass sheet 120a is generally centered over the mold surfaced 101a and the port mold surface 130 during the positioning step.

The method of making a bio-container further includes a step of heating the mold 110a and the glass sheet 120a to a molding temperature above or about equal to the softening point of the glass sheet for a time long enough to ensure that there is substantial uniformity in the temperature within the glass sheet 120a near or about the molding temperature. For example, in one exemplary implementation, a furnace with proportional-integral-derivative (PID) control can be employed such that the PID control brings the temperature to the final, desired set point without over-shooting more than 10 degrees in about 5 minutes. This timing allows sufficient temperature equilibrium of the glass sheet 120a for a given set point. Accordingly, the mold 110a and the glass sheet 120a are situated in a furnace, oven or other heating structure prior to the initiating of this step. Upon completion of the heating step, e.g., through PID control, a de-pressurizing step is conducted in which a vacuum is applied to the plurality of vacuum holes 140 in the mold 110a for a relatively short duration, typically from about 0.5 to about 5 minutes. The PID controller slows the ramp rate of the furnace when nearing the final set point temperature to avoid any temperature overshoots, typically with a duration of up to about 5 minutes over the last few degrees before reaching the set point temperature. This vacuum through the holes 140 pulls the glass sheet 120a toward the mold surface 101a and the port mold surface 130 to form the glass sheet 120a into a container half 20a, 20b (see FIGS. 3B and 3C). After formation of the glass sheet 120a into a container half 20a, 20b, the container half is preferably removed from the mold while remaining at a relatively high temperature (e.g., about 300° C. to 500° C. to ensure that the container half 20a, 20b can be readily removed from the mold 110a without damage, breakage or other defects. Conversely, if the container half 20a, 20b is removed at too low of a temperature (e.g., from room temperature to 200° C.), the mold 110a may contract more than the container half and seize the container half to the mold 110a.

Suitable materials for construction of the mold 110a include metals, metal alloys, graphite, boron nitride, and other refractory ceramic materials. In general, the mold 110a should be fabricated from a material or materials capable of withstanding the temperatures associated with heating the glass sheet 120a at or above its softening point. Depending on the material or materials selected for the mold 110a, it may be machined, fired or otherwise processed to obtain the desired design, including the mold surface 101a and port mold surface 130 as readily understood by those skilled in the field. As necessary, an inert atmosphere (e.g., argon gas, nitrogen gas, helium gas, and mixtures of these gases) can be employed during the heating and de-pressurizing steps for a method employing a mold fabricated from an atmospheric sensitive material, such as graphite.

According to a further aspect of the foregoing method of making a bio-container (e.g., bio-container 100, 100a), the vacuum holes 140 are configured in the mold surface 101a and port mold surface 130 such that the vacuum applied to the holes 140 during the de-pressurizing steps is individually controlled or controllable in these regions. In some configurations of the mold surface 101a and port mold surface 130, the geometries are such that increased or decreased vacuum levels applied to particular regions of the mold 110a can improve the formation of the container halves 20a, 20b. For example, the smaller surface area of the port mold surface 130 relative to the mold surface 101a can require the glass sheet 120a to conform to relatively small radius bends, which require more force to overcome the surface tension of the glass at a constant viscosity. As it is preferable to maintain an isothermal condition over the mold 110a during the heating and de-pressurizing steps, selective heating of the glass sheet 120a in proximity to the port mold surface 130 is less-desirable. Instead, the preferred approach is to increase the vacuum level through the vacuum holes 140 within the port mold surface 130 to ensure that the ports 30 in the container halves 20a, 20b (see FIGS. 3B, 3C) have their required shape after the de-pressurizing step.

With further regard to the mold 110a (see FIGS. 3, 3A) employed in the method of making a bio-container, the same mold 110a can be employed to fabricate identical or substantially similar container halves 20a, 20b in terms of geometry and features that can subsequently be sealed to form a bio-container 100a (or bio-container 100 as shown in FIGS. 1-1D). In another aspect, multiple molds 110a can be employed according to the foregoing method with differing mold surfaces 101a and/or port mold surfaces 130 to fabricate container halves with differing geometries, thickness, port locations, and other features. These container halves possessing differing geometries (e.g., one container half with a set of integral ports 30 in a particular location and a second container half with a set of integral ports 30 in a different location) can then be sealed to form a bio-container.

After the glass sheet 120a has been formed into a container half 20a, 20b, the resulting container half can be annealed as part of the method of making a bio-container. The annealing step can be conducted in a separate annealing furnace (not shown) after the container half 20a, 20b has been removed from the mold 110a. In another aspect, the container half 20a, 20b is annealed in place by lowering the temperature of the furnace, oven or the like that houses the mold 110a. In preferred implementations of the method, the optional annealing step is conducted prior to any sealing of the container halves 20a, 20b.

After the container halves 20a, 20b (see FIGS. 3B and 3C) have been formed according to the foregoing method, the method further includes a step of sealing the container halves 20a, 20b to form the bio-container 100a as shown in FIG. 3D (or bio-container 100 as shown in FIGS. 1-1D). In particular, the container halves 20a, 20b are sealed along a seam 50 to form the bio-container, e.g., bio-container 100a. Accordingly, the bio-container 100a formed according to the method includes a single-use container 20 defined by its two container halves 20a, 20b and a seam 50. As shown in FIG. 3D, the single-use container 20 includes an exterior surface 22, an interior surface 24 and a container thickness 16 (see FIG. 2A), typically from about 0.2 mm to about 2 mm. Further, the single-use container 20 includes at least one port 30 (e.g., as formed from the port mold surface 130).

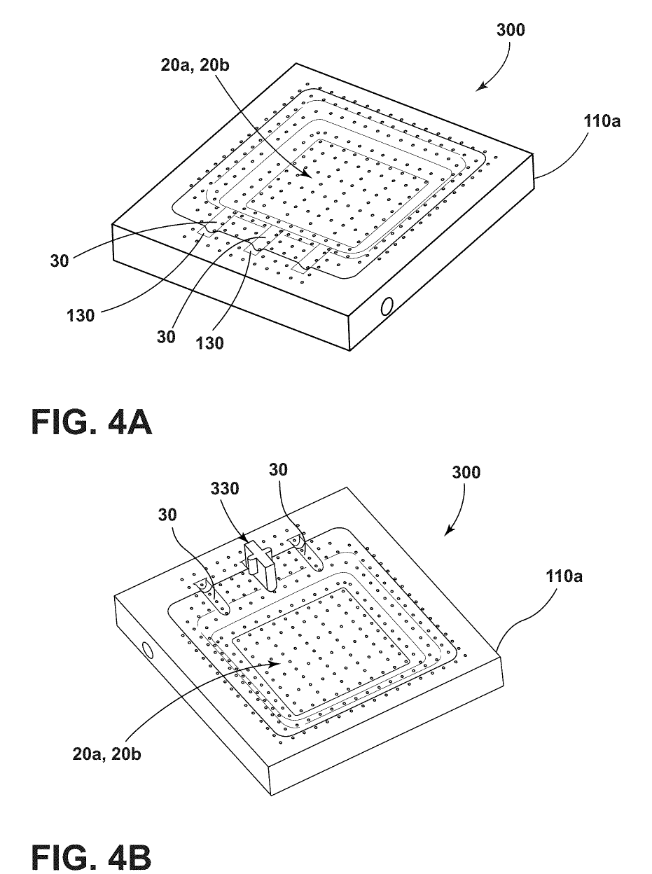

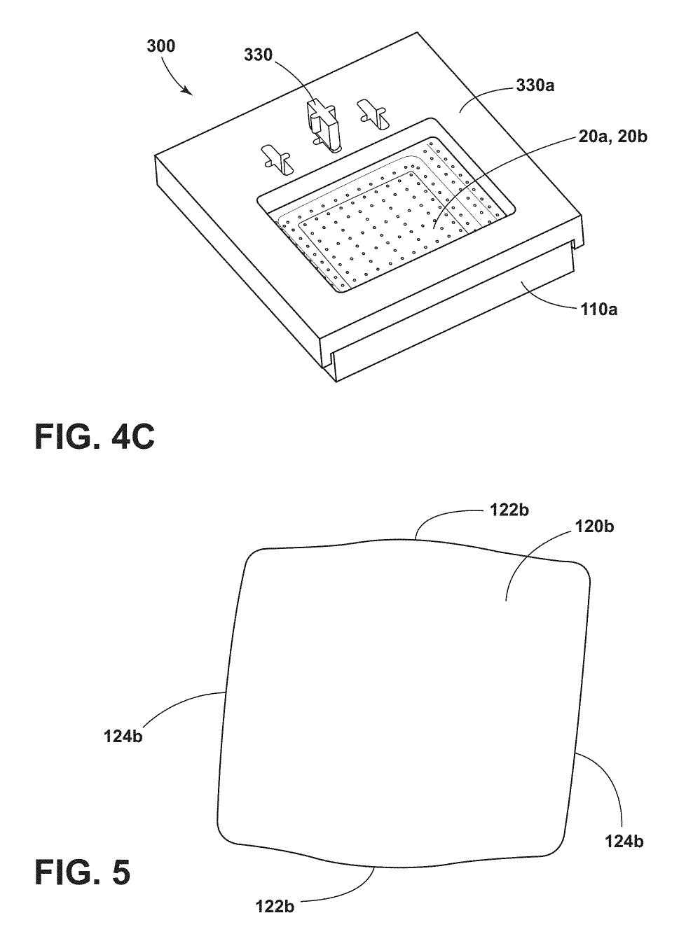

Referring again to FIGS. 3-3D, one example implementation of the foregoing method of making a bio-container was conducted as follows. A pre-cut, laminated glass sheet 120a having a width of 280 mm, a length of 240 mm and a thickness 126 of about 0.7 mm was positioned on the mold surface 101a of a mold 110a. In some embodiments, the mold 110a is machined from an Inconel® metal alloy (or a substantially similar alloy with regard to the relevant properties) and coated with a graphite spray release agent. Further, the laminated glass sheet 120a includes an outer clad layer 41, inner clad layer 42 and a core layer 43 (see FIG. 1C). According to this method, a vacuum line was connected to the plurality of vacuum holes 140 in the mold 110a and a mechanical vacuum pump. At this stage of the method, argon gas was purged into a furnace housing the mold 110a and the glass sheet 120a positioned thereon at a flow rate of about 20 lpm. More particularly, the glass sheet 120a was positioned such that it covered all of the vacuum holes 140 and a graphite frame (not shown, but comparable to frame 330a depicted in FIG. 4C) was placed over the sheet to ensure that the sheet did not bend during the subsequent heating and de-pressurizing steps. As part of the heating step, the mold 110a and the sheet 120a were heated to about 800° C. at a heating rate of about 10° C./min. Once the furnace reached 800° C., a vacuum valve was opened prior to any hold time at the 800° C. set point and the mold 110 was de-pressurized by the mechanical vacuum pump connected to the plurality of vacuum holes 140. In particular, the de-pressurizing step was conducted at a pressure of about −10 torr (about −1300 Pa or 0.013 atmospheres) and allowed to run for about 1.5 minutes. The mechanical vacuum pump was then switched off, and the furnace holding the glass sheet 120a and the mold 110a was ramped down to about 600° C. to 650° C. This cooling step was conducted at a cooling rate faster than the natural cooling rate of the furnace by opening the furnace door and switching off power to the furnace. At about 600° C., the furnace door was closed and the remainder of the cooling was conducted according to a natural furnace cooling rate. By slowing the cooling rate down in this regime by relying on only the natural cooling rate of the furnace (i.e., without any additional cooling), the now-formed container half 20a, 20b was effectively annealed as it was cooled toward an ambient temperature. Finally, the container half 20a, 20b was removed from the mold 110a upon cooling to a temperature suitable for handling (e.g., about 300° C. or less).

According to another aspect of the method of making a bio-container, fabrication of the ports 30 in the container half 20a, 20b can be augmented with a die pressing operation 300, as schematically depicted in FIGS. 4A-4C. As shown in FIG. 4A, a container half 20a, 20b that was fabricated within the mold 110a can be subjected to an additional die pressing step to augment the formation of the ports 30. In some aspects, the die pressing operation is separately conducted after the heating and de-pressurizing steps of the method of making a bio-container. In particular, the container half 20a, 20b and mold 110a may require additional heating such that the container half 20a, 20b is brought to a temperature at or above the softening point of the glass composition of the halves 20a, 20b prior to the die pressing operation 300. Alternatively, the container half 20a, 20b can be maintained at these temperatures after the de-pressurizing steps. More particularly, the container half 20a, 20b depicted in FIG. 4A has been formed such that it conforms to the mold surface 101a and port mold surface 130 (see FIG. 3), but in a configuration such that the ports 30 formed in the mold surface 130 require additional processing. For example, geometries of the ports 30 with tight bend radii may necessitate additional mechanical processing before or after the de-pressurizing step to ensure that the glass sheet well-conforms to the port mold surface 130. Accordingly, the ports 30 of the container half 20a, 20b can be further formed in the port mold surface 130 by subjecting the container half 20a, 20b to direct pressure from a die 330 as shown in FIG. 4B. In particular, the die 330 is mechanically pressed (e.g., by a robot, human or other fixture) against the container half 20a, 20b to form the ports 30 over the port mold surface 130. In addition, the die 330 can be guided during the die pressing step 300 by a frame 330a as shown in FIG. 4C. The frame 330a is machined or otherwise manufactured to fit over the outer perimeter and edges 150 (see FIG. 3) of the mold 110a to properly align the die 330 with the port mold surface 130. In some implementations, the dies 330 and the frame 330a are fabricated with cross-members and cross-slots, respectively, to improve alignment of the die 330 in the port mold surface 130 during the die pressing operation 300.

According to some aspects of the die pressing operation 300 (see FIGS. 4A-4C), a weight (not shown) is added on the top surface of each die 330 to provide a static force that presses down on the glass sheet. In this implementation of the die pressing operation 300, the use of a static force applied by the die 330 allows the operation to be conducted during the heating and de-pressurizing steps of the method of making a bio-container. That is, the die pressing operation 300 can augment the formation of the ports 30 by providing an additive static force in the port mold surface 130 in addition to the vacuum force provided through the vacuum holes 140 during the de-pressurizing. As such, the die pressing operation 300, in some embodiments, can be conducted simultaneously with the heating and de-pressurizing steps, thus simplifying the overall method of making the bio-container and reducing process costs. Another advantage of the die pressing operation 300, in combination with the de-pressurizing step, is that it can be employed to create complex container half 20a, 20b geometries, while allowing for substantially isothermal processing.

According to another implementation of the method of making a bio-container, particular geometries of the glass sheet (see FIG. 3A, glass sheet 120a) can be employed to facilitate the development of the container half 20a, 20b. As a rectangular glass sheet (e.g., glass sheet 120a) is formed into the three-dimensional shape of the container half 20a, 20b during the heating and de-pressurizing steps, the glass is pulled down into the mold surface 101a and port mold surface 130 (see FIG. 3A). As the glass is viscoelastic, the sides and/or edges of the container half 20a, 20b can end up in an undesirable concave shape. Further, the walls of the container half 20a, 20b can be subjected to thinning if the edges of the glass sheet are pinned or otherwise held during the heating and de-pressurizing steps. As shown in FIG. 5, a shaped glass sheet 120b can be configured for forming a container half 20a, 20b to solve these problems associated with certain glass sheet 120a and container half 20a, 20b geometries. In this configuration of the glass sheet 120b (see FIG. 5), the edges 122b and 124b are configured with a convex shape. As the glass sheet 120b is formed into the container half 20a, 20b according to the foregoing method of making a bio-container, the convex edges 122b, 124b of the glass sheet 120b are drawn inward; consequently, the container half 20a, 20b is formed with significantly straighter edges. One advantage of employing a shaped glass sheet 120b is that it can be configured to largely or completely eliminate the need for a down-stream trimming operation (e.g., the trimming operation 400 outlined below) to the container half 20a, 20b.

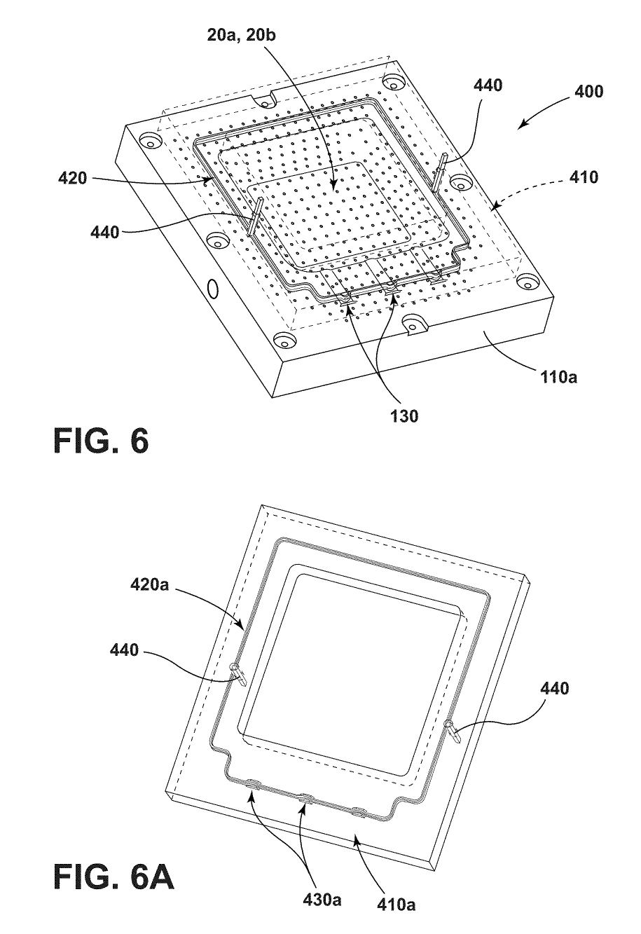

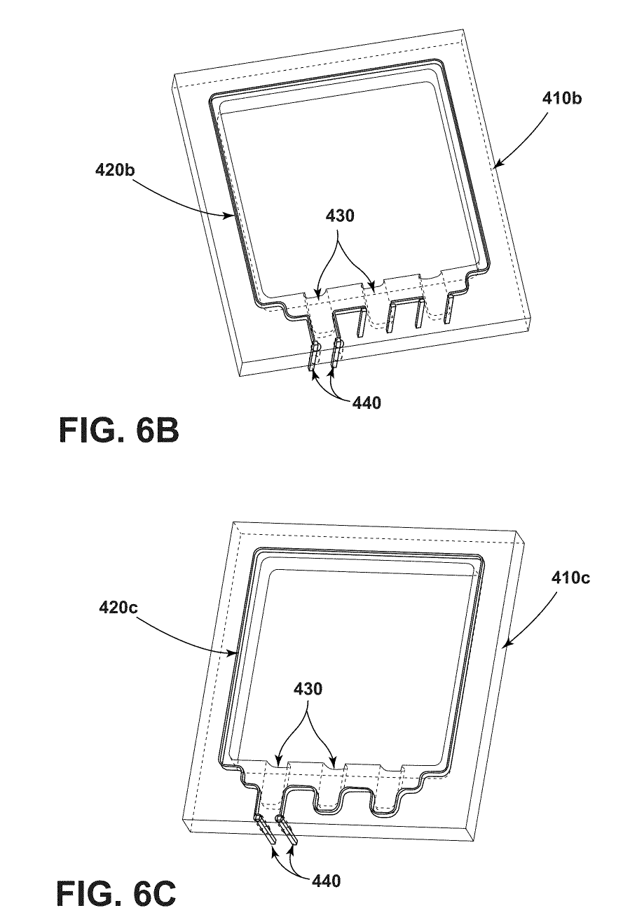

According to another embodiment of the method of making a bio-container, the container half 20a, 20b can be subjected to a trimming operation 400 after the heating and de-pressurizing steps (and the optional die pressing operation 300 shown in FIGS. 4A-4C), as schematically depicted in FIG. 6. A trimming operation 400 in the formation of the container half 20a, 20b may be needed because of the existence of excess edges and regions of the initial glass sheet 120a that overlap the mold surface 101a and/or port mold surface 130 after the heating and de-pressurizing steps. In some aspects of the trimming operation 400 depicted in FIG. 6, the container half 20a, 20b is heated again to a temperature at or above the strain point of its glass composition. At this point of the trimming operation 400, a trim assembly 410 (e.g., a block fabricated from a non-conductive ceramic or refractory block) is seated over the mold 110a and the container half 20a, 20b. The trim assembly 410 may, in some embodiments, include an electrical lead configuration 420 alloy connected to a plurality of electrodes 440. In some embodiments, the electrical lead configuration 420 is an electrically resistive strip about 0.5 to 2 mm thick and about 3 to 10 mm wide that is fabricated from a platinum alloy. Other configurations of the electrical lead configuration 420 are viable, as understood by those with ordinary skill in the field, provided that they have the capability of sufficiently heating the glass compositions of the disclosure to effect trimming.

After the container half 20a, 20b has been heated to a temperature at or above its strain point, the electrical lead configuration 420 and electrodes 440 are then employed to directly heat the container half 20a, 20b to a temperature at or above the softening point of the glass composition of the container half 20a, 20b. The electrical lead configuration 420 then locally heats the container half 20a, 20b at a specific area to soften the glass, and the weight of the trim device 410 separates the glass at this location. As the glass is separated, the excess edges and regions of the container half 20a, 20b can be mechanically removed and separated from the container half 20a, 20b. An advantage of heating the container half 20a, 20b to a temperature near its strain point prior to, or during, the trimming operation is that it minimizes the likelihood of thermal shock in the container half 20a, 20b during its direct heating by the electrical lead configuration 420.

FIGS. 6A, 6B and 6C depict various trim assembly configurations that can be employed in the trimming operation 400 shown in FIG. 6. Referring to FIG. 6A, a trim assembly 410a with an electrical lead configuration 420a, 430a is depicted that can be employed in the trimming process to trim material around the circumference and in the port regions of a container half as part of the method of making a bio-container. In particular, the trim assembly 410a can be employed to trim excess glass material from the periphery of the container half 20a, 20b and any excess glass material in the openings of the ports 30 (see FIG. 3D). As shown in FIG. 6A, the electrical lead configuration 430a has extended convex-shaped appendages (e.g., in proximity to the port mold surface 130 as shown in FIG. 6) to trim excess glass not otherwise formed in the port mold surface 130 during the de-pressurizing step and optional die pressing operation 300. In addition, this configuration of the trim assembly 410a leaves webbing between the ports 30, which can improve the overall strength and integrity of the container half 20a, 20a and, ultimately, the bio-container 100, 100a formed from these halves.

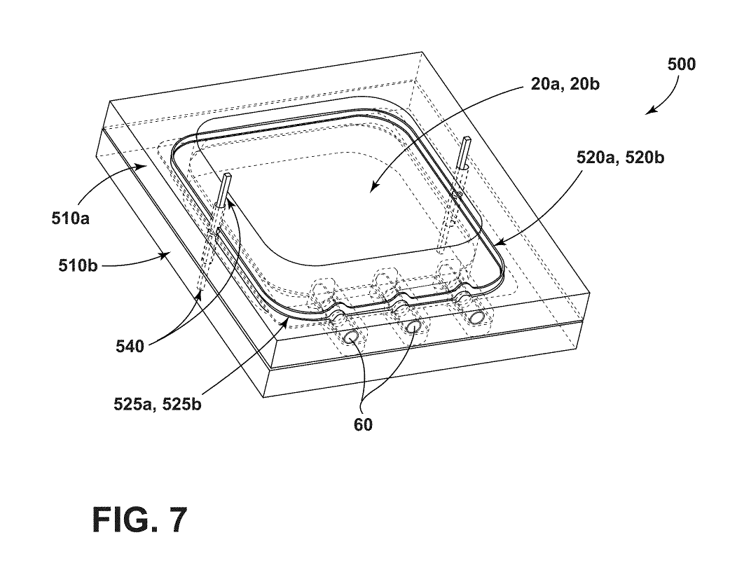

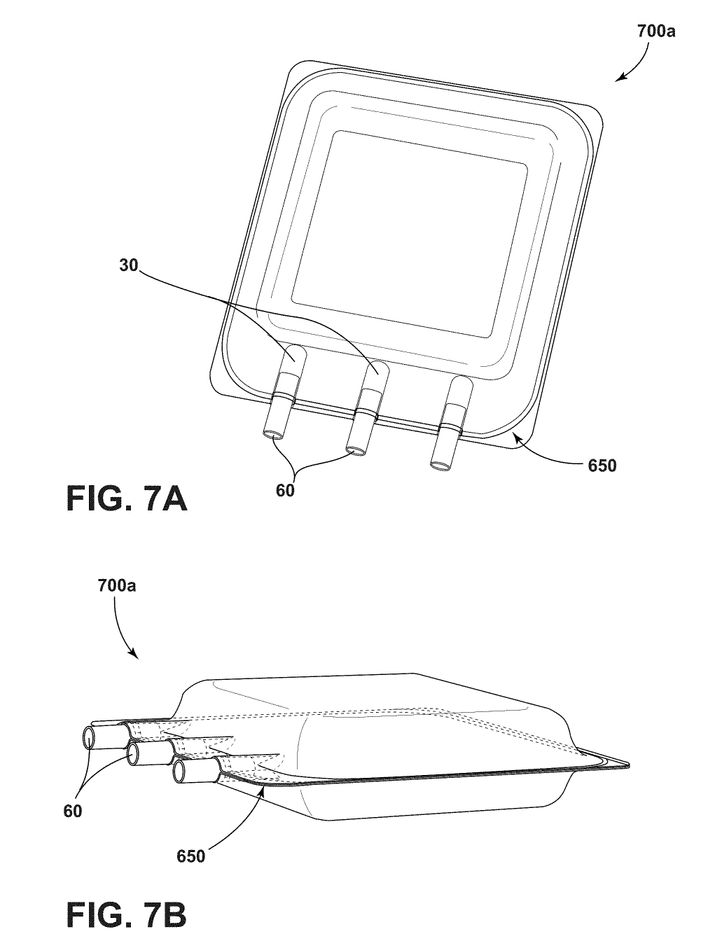

Referring to FIG. 6B, a trim assembly 410b with an electrical lead configuration 420b is depicted that can be employed in the trimming process to trim material around the circumference and in the port regions of a container half as part of the method of making a bio-container. In particular, the trim assembly 410b can be employed to trim excess glass material from the periphery of the container half 20a, 20b and any excess glass material in regions between the ports 30 (see FIG. 3D). As shown in FIG. 6B, the electrical lead configuration 420b has extended appendages configured to trim excess glass between the port regions 430 of the trim assembly 410b. Note that these port regions 430 in the trim assembly 410b are configured to substantially correspond to the port mold surface 130 of the mold 110a (see FIG. 6). Further, the trim assembly 410b depicted in FIG. 6B facilitates the trimming of excess glass material between the ports 30, but largely leaves any glass material in the ports 30 untouched. As such, a container half 20a, 20b trimmed with a trim assembly 410b may include additional tubes 60 inserted into the ports 30 to complete the formation of the bio-container (see FIG. 7A). Referring to FIG. 6C, a trim assembly 410c is depicted with largely the same construction and function as the trim assembly 410b depicted in FIG. 6B. A difference is that electrical lead configuration 420c in the trim assembly 410c relies on only two electrodes 440, whereas the trim assembly 410b (see FIG. 6B) relies on many more electrodes 440 (or jumpers). However, the trim assembly 410c also removes some material in the ports 30, which may be useful in some geometries of the container half 20a, 20b subjected to the trimming operation 400.

According to another aspect of the method of making a bio-container, the sealing step can be conducted according to a direct sealing operation 500 as depicted in FIGS. 7, 7A and 7B. Referring to FIG. 7, the direct sealing operation 500 can employ upper and lower sealing blocks 510a, 510b, each with electrical lead configurations 520a, 520b. The electrical lead configurations 520a, 520b are each configured with electrodes 540 and, collectively, they are comparable in structure and function to the electrical lead configuration 420, 420a-c and electrodes 440 employed in the trimming operation 400 (see FIG. 6, 6A-6C). During the direct sealing operation 500, the sealing blocks 510a, 510b are fitted over a pair of container halves 20a, 20b, such that sealing portions 525a, 525b around their periphery are in substantial contact with one another. In some aspects, the sealing portions 525a, 525b are peripheral or other portions of the halves 20a, 20b that, upon subsequent processing, form a joint or seam (e.g., seam 50 as shown in FIG. 2A, or seam 650 as shown in FIGS. 7A and 7B). Similar to the trimming operation 400, the sealing operation 500 includes heating the container half 20a, 20b again to a temperature at or above the strain point of its glass composition. At this point, the electrical lead configurations 520a, 520b of the sealing blocks 510a, 510b are then employed to directly heat the sealing portions 525a, 525b of the container half 20a, 20b to a temperature at or above the softening point of the glass composition of the container half 20a, 20b. Preferably, the sealing portions 525a, 525b of the container half 20a, 20b are heated to no more than 200° C. greater than the softening point of the glass composition. The sealing portions 525a, 525b are now pressed together, by the natural weight of the upper container half 20a or by an additional weight (not shown) placed over the upper container half 20a.

After the sealing portions 525a, 525b are joined by virtue of the direct sealing operation 500, a bio-container 700a is now formed having a seam 650 (see FIGS. 7A and 7B) between the container halves 20a, 20b. The bio-container 700a is then cooled to an ambient temperature and then removed from the sealing blocks 510a, 510b. Further, according to some embodiments of the direct sealing operation 500 depicted in FIGS. 7-7B, a bio-container having additional tubes 60, as-sealed in the ports 30, can be formed. These tubes 60 can be pre-fabricated or otherwise cut to size from soda-lime glass, borosilicate glass, quartz, silica, glass materials comparable in composition to those employed for the container halves 20a, 20b, and other glass-ceramic or ceramic materials with a similar or higher softening point than the glass composition employed in the container halves 20a, 20b. In particular, the tubes 60 are inserted into the ports 30 prior to the container halves 20a, 20b being enclosed by the sealing blocks 510a, 510b. As the sealing portions 525a, 525b of the container halves 20a, 20b are directly heated by the electrical lead configurations 520a, 520b, the ports 30 are also heated such that they are sealed around the tubes 60. As a result, bio-container 700a is formed from the container halves 20a, 20b and tubes 60 in essentially one sealing operation 500. More generally, an advantage of the direct sealing operation 500 for forming a bio-container, e.g. bio-container 700a, is that it does not require the addition of any other structures and/or sealing materials to the seam 650 of the bio-container. Accordingly, a direct-sealed bio-container 700a can be configured without significant concerns over leaching of materials associated with the seam 650 between the container halves 20a, 20b.

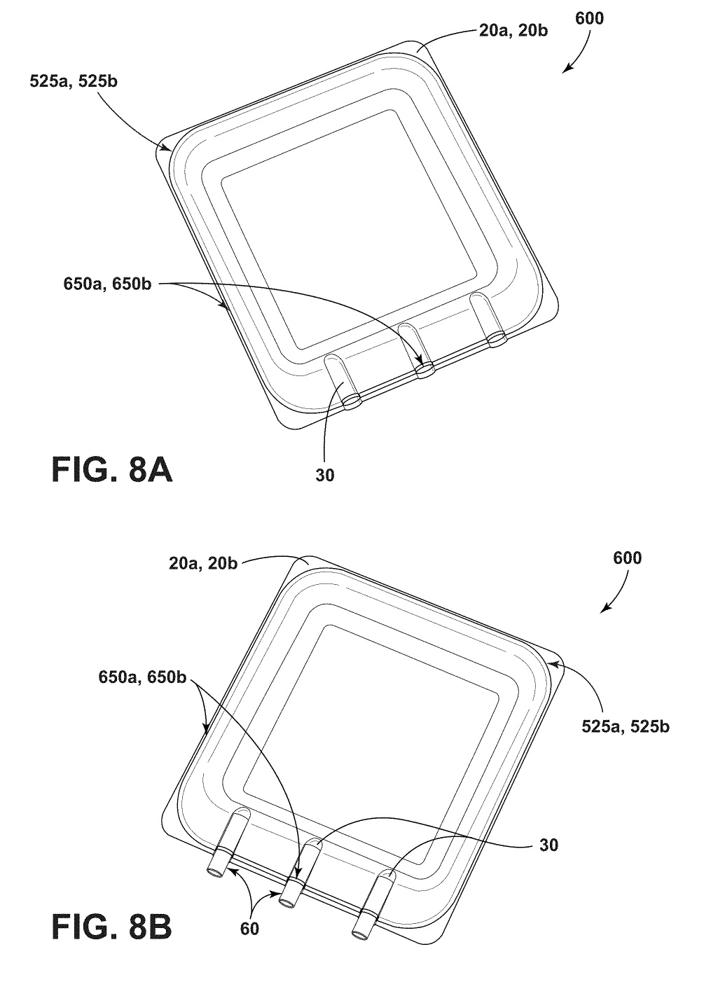

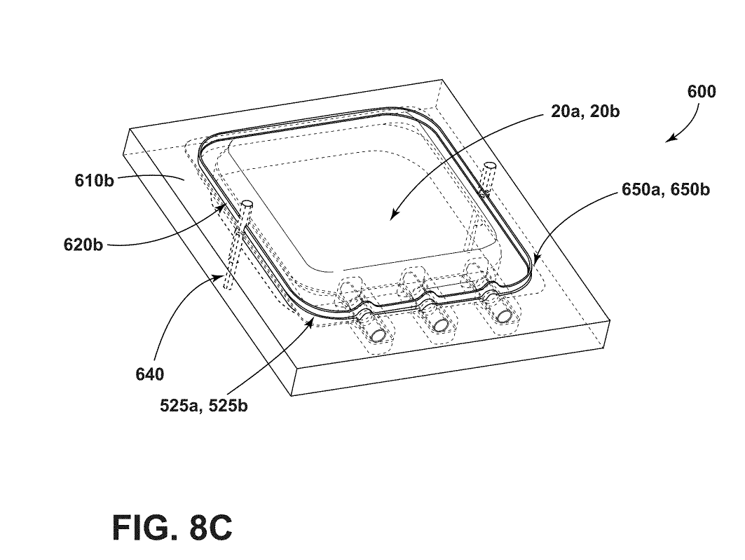

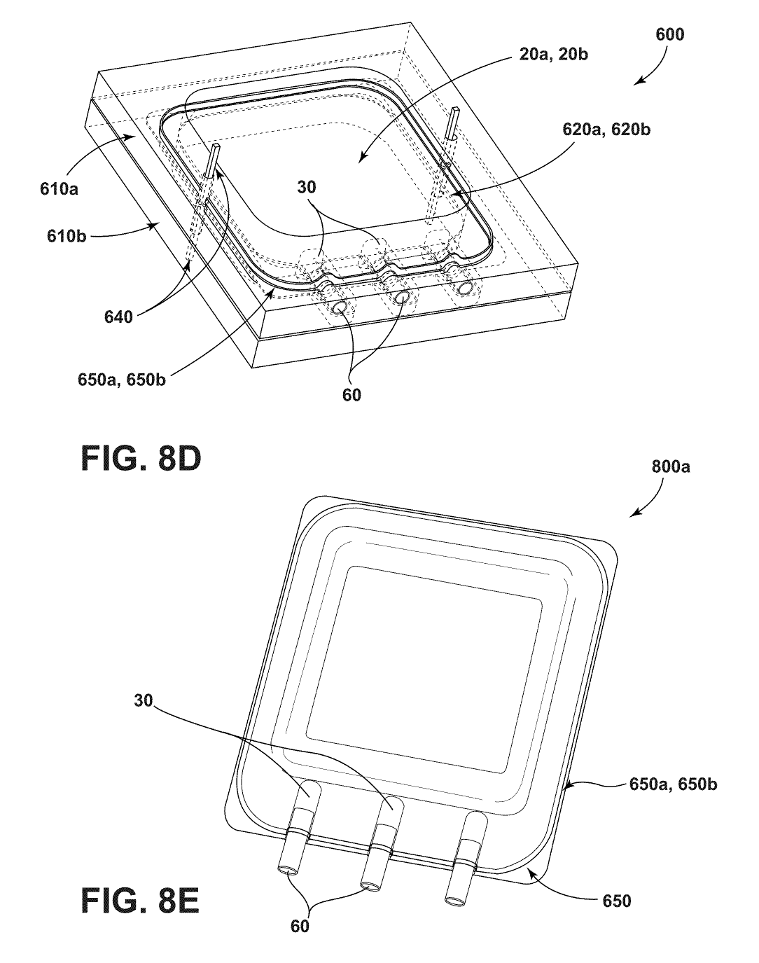

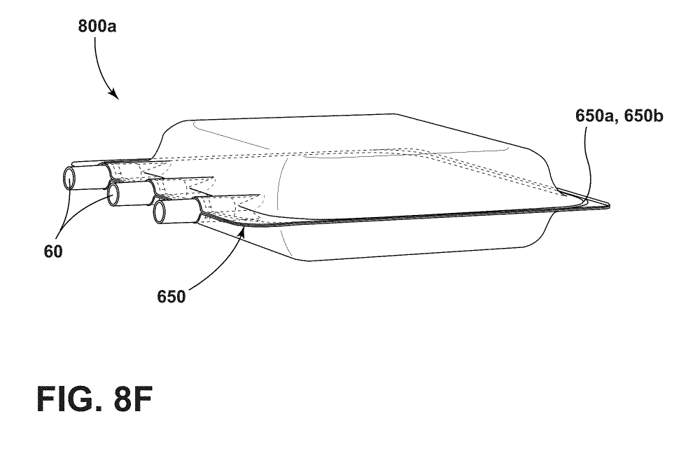

According to a further aspect of the method of making the bio-container, the sealing step can be conducted according to a frit sealing operation 600 as depicted in FIGS. 8A-8D. Referring to FIG. 8A, the frit sealing operation 600 first involves a step of applying a frit 650a to the sealing portion 525a of a container half 20a and a frit 650b to a sealing portion 525b of the container half 20b. The frit 650a 650b can possess a glass or glass-ceramic composition, typically ground to a particular particle size distribution and suspended in an organic binder and/or paste. The composition of the frit 650a, 650b can be any of a variety of glass and glass-ceramic compositions. Preferably, a silicate-based glass with no leachable components, as assessed relative to a Permitted Daily Exposure (PDE) level, is employed for the frit 650a, 650b. In certain embodiments, the frit 650a, 650b should soften below the softening temperature associated with the glass composition for the container halves 20a, 20b. According to another embodiment of the frit sealing operation 600, the frit 650a, 650b is selected to have a CTE of no more than ±20% different than the CTE of the glass composition employed for the container half 20a, 20b. Preferably a CTE difference between the frit 650a, 650b and the container half 20a, 20b of no more than ±10% is maintained. An example glass frit composition employed for the frit 650a, 650b is as follows: 39.94 wt. % (43.4 mol %) SiO₂; 37.64 wt. % (35.3 mol %) B₂O₃; 8.52 wt. % (9.0) Na₂O; 6.20 wt. % (4.30 mol %) K₂O; 6.23 wt. % (5.00 mol %) ZnO; and 1.37 wt. % (3.00 mol %) Li₂O.

Typically, the frit 650a, 650b employed in the frit sealing operation 600 is in the form of a paste at ambient temperature and can be dispensed into or otherwise on the sealing portions 525a, 525b of the container half 20a, 20b using various dispensing equipment as understood by those with ordinary skill in the field of the disclosure. An exemplary frit composition suitable for the method can be fabricated by mechanically grinding glass, according to the composition noted earlier, and screening it through a 400 mesh sieve. The resultant particle size distribution has about 50% of the particles at about 8 microns in size. The formulation of the frit paste is about 75 wt. % glass, 23 wt. % butyl carbatol acetate and about 2 wt. % Dow Corning® silicone fluid.

As also depicted in FIG. 8A, the frit 650a, 650b can be applied to the ports 30 of the container half 20a, 20b, to facilitate the placement and later sealing of the tubes 60 into the ports 30 (see FIG. 8B). In other aspects of the method in which a bio-container with ports lacking tubes is fabricated, the frit 650a, 650b is purposely masked or otherwise not dispensed in the ports 30 of each of the container halves 20a, 20b. According to another embodiment, the frit 650a, 650b is applied to the sealing portions 525a, 525b of the container halves 20a, 20b while the tubes 60 are already positioned in the ports. In this approach, the application of the frit over the tubes 60 is optional prior to joining the two container halves 20a, 20b together.

After the frit 650a, 650b is applied according to the frit sealing operation 600, the container halves 20a, 20b can be joined. In some embodiments, the joining can be conducted by positioning the container halves 20a, 20b together, thereby joining the halves 20a, 20b at the sealing portions 525a, 525b containing the frit 650a, 650b. At this point, the container halves 20a, 20b, as joined by the frit 650a, 650b, are heated to remove any organic materials in the frit. A typical thermal schedule for removing the organics in the frit is to heat the container halves 20a, 20b to about 350° C. at a heating rate of about 5° C./min, and holding for about one hour or longer to remove the organics in the frit. A next step in the frit sealing operation 600 is to fuse the frit 650a, 650b to the sealing portions 525a, 525b of the container halves 20a, 20b. The fusing step, in some embodiments, can include heating the container halves 20a, 20b and the frit 650a, 650b to about 650° C. at a heating rate of about 5° C./min, holding for about an hour, and then cooling the container halves 20a, 20b down to ambient temperature at a typical furnace cooling rate (e.g., the natural rate of cooling the part when the power has been shut off to the furnace). Upon cooling, the container halves 20a, 20b are then joined with frit 650a, 650b at a seam 650 to form a frit-sealed bio-container 800a (see FIGS. 8E and 8F). Further, the tubes 60 are joined within the ports 30 by frit 650a, 650b or otherwise secured by the seam 650 of the container halves 20a, 20b containing frit 650a 650b in embodiments in which no frit is applied to the ports 30 and/or the tubes 60.

As the foregoing embodiment of the frit sealing operation 600 involves isothermally heating the container halves 20a, 20b and the frit 650a, 650b, it can be important to select a frit composition with a relatively low softening point (e.g., as compared to the softening point of the glass composition selected for the container halves 20a, 20b and/or inner clad layer 42, as applicable and as shown in FIGS. 1-1D) when employing this approach. In particular, a frit 650a, 650b with a relatively low softening point will ensure that the container halves 20a, 20b do not deform during the frit sealing operation 600. Nevertheless, other embodiments of the frit sealing operation 600 can seal container halves 20a, 20b with compositions for the frit 650a, 650b having higher softening points using the frit sealing apparatus depicted in FIGS. 8C and 8D. For example, certain frit compositions with higher softening points may be needed to ensure proper CTE matching with the particular glass composition selected for the container halves 20a, 20b.

More particularly, the single-block frit sealing apparatus 610b shown in FIG. 8C or dual-block frit sealing apparatus 610a, 610b shown in FIG. 8D can be employed by the frit sealing operation 600 to directly heat the frit 650a, 650b, without risk of distortion to the container halves 20a, 20b during the fusing step. The single-block frit sealing apparatus 610a, 610b are largely identical to the sealing blocks 510a, 510b employed in the direct sealing operation 500 (see FIG. 7). In particular, the frit sealing apparatus 610a, 610b each include an electrical lead configuration 620a, 620b configured with electrodes 640. During the steps of removing the organics and fusing the frit 650a, 650b, the single-block frit sealing apparatus 610b is fitted beneath a pair of container halves 20a, 20b, such that the sealing portions 525a, 525b of the halves 20a, 20b and the frit 650a, 650b around their periphery are in substantial contact with one another as shown in FIG. 8C. Similarly, the dual-block frit sealing apparatus 610a, 610b can be fitted around the pair of container halves 20a, 20b as shown in FIG. 8D. At this point, the frit sealing operation 600 involving a single-block or dual-block frit sealing apparatus 610a, 610b includes heating the container half 20a, 20b again to a temperature at or above the strain point of its glass composition. At this point, the electrical lead configuration 620a, 620b of the sealing apparatus 610a, 610b are then employed to directly heat the sealing portions 525a, 525b of the container half 20a, 20b containing the frit 650a, 650b to a temperature at or above the softening point of the frit 650a, 650b such that the sealing portions 525a, 525b are fused together by the frit 650a, 650b. The fusing step, in some embodiments, can include directly heating the sealing portions 525a, 525b of the container halves 20a, 20b and the frit 650a, 650b to about 650° C. at a heating rate of about 5° C./min, holding for about an hour, and then cooling the container halves 20a, 20b down to ambient temperature at a typical furnace cooling rate (e.g., the natural rate of cooling the part when the power has been shut off to the furnace). Upon cooling, the container halves 20a, 20b are now joined with frit 650a, 650b at a seam 650 to form a frit-sealed bio-container 800a (see FIGS. 8E and 8F). Further, the tubes 60 are joined within the ports 30 by frit 650a, 650b or otherwise secured by the seam 650 of the container halves 20a, 20b containing frit 650a 650b in embodiments in which no frit is applied to the ports 30 and/or the tubes 60.

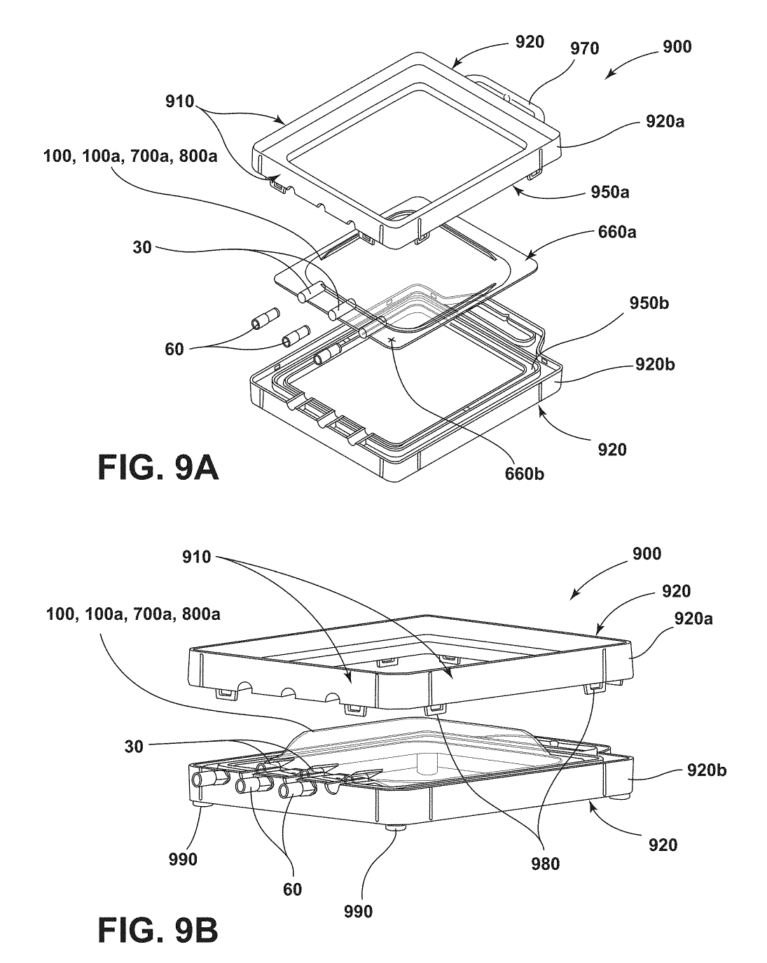

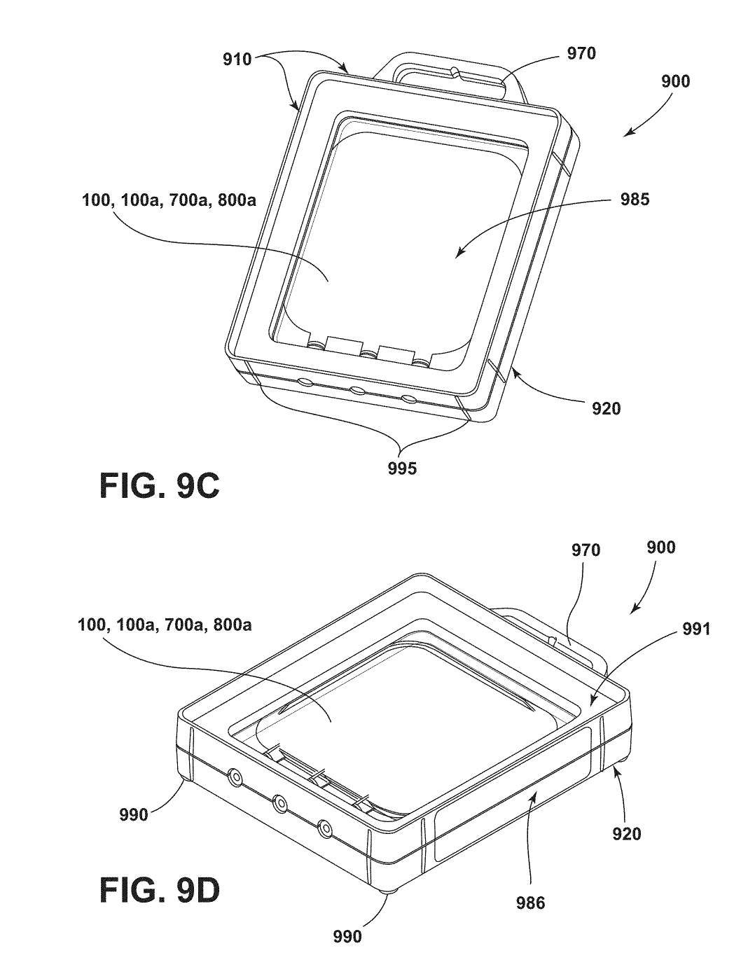

Referring now to FIGS. 9A-9D, a bio-container assembly 900 is depicted according to a further aspect of the disclosure. The bio-container assembly 900 can, in some implementations, include a bio-container 100, 100a, 700a, 800a (see FIGS. 1, 2, 7A, 8E) having an interior surface, an exterior surface and a container thickness (e.g., interior surface 24, exterior surface 22 and container thickness 16 of the bio-container 100a depicted in FIG. 2). Further, such a bio-container of the assembly 900 can include at least one port and/or tube that is coupled to the bio-container (e.g., at least one tube 60 of the bio-container 100a depicted in FIG. 2).

Referring again to FIGS. 9A-9D, the bio-container assembly 900 also includes a holder element 920 with upper and lower halves 920a, 920b that comprise a polymeric material. Various polymeric materials are suitable for the halves 920a, 920b including, but not limited to, polystyrene, acrylonitrile butadiene styrene and polycarbonate. In addition, the bio-container assembly 900 includes a cushion 950a, 950b affixed to the upper and lower halves 920a, 920b, respectively, for cushioning one or more exterior surfaces of the bio-container 100, 100a, 700a, 800a (e.g., exterior surfaces 22 of the bio-container 100a depicted in FIG. 2). Further, the cushion 950a, 950b and the holder element 920, including its upper and lower halves 920a, 920b, are configured to enclose the bio-container.

The bio-container assembly 900 has all of the functions, benefits and advantages of its bio-containers, as outlined earlier in the disclosure. Further, the addition of the holder element 920 to the bio-container as part of the assembly 900 allows a user to more effectively transport and use the bio-container with less concern over damage to the bio-container and contamination to the contents (e.g., biological, pharmaceutical and bio-pharmaceutical end products, cell cultures, biologics, etc.) within it. That is, the holder element 920 of the bio-container assembly 900 can act as an additional safety and/or barrier to the bio-container. In addition, the rigidity and structural features of the holder element 920 improve the ease of handling and transport of the bio-container and the contents within it, as the bio-container assembly 900 can be stacked and handled with relative ease. Still further, the cushioning afforded by the holder element 920 to the bio-container ensures that the bio-container does not fracture or otherwise fail from rough handling, contact, and the like. In addition, the added cushioning offered by the holder element 920 can, in some implementations, enable bio-container configurations made from glass sheets without compressive stress regions or limited compressive stress regions. One benefit of not requiring a compressive stress region (or a limited compressive stress region) is a reduction in overall manufacturing costs for the bio-container assembly 900. It should also be understood that the holder element 920, and the concepts associated with it in the disclosure, can serve as an enabling technology for the bio-containers of the disclosure, with all of the foregoing benefits and advantages.

As depicted in FIGS. 9A-9D, the holder element 920 according to some implementations can include an upper and lower half 920a, 920b. The holder element 920 may also include bar-coding, labels and other information at a designated location 986 (see FIG. 9D) in certain embodiments. In other aspects, the holder element 920 is fully enclosed (not shown) such that the bio-container 100, 100a, 700a, 800a is not visible, but is fully-encased for maximum impact protection within the holder element 920. In an additional implementation, the holder element 920 can be fabricated from a substantially translucent, polymeric material to afford maximum impact protection of the holder element 920 while maintaining visibility to the contents within the bio-container 100, 100a, 700a, 800a. In other implementations, the holder element 920 may include a window 985 (see FIG. 9C) or is otherwise configured such that it only partially encloses the bio-container 100, 100a, 700a, 800a. In the latter case, the holder element 920 can configured as mesh or screen (not shown) to offer impact protection while offering some visibility to the bio-container 100, 100a, 700a, 800a and the contents within it. Ultimately, the incorporation of a window 985 or the use of a translucent polymeric material for the holder element 920 can facilitate viewing of the contents within the bio-container enclosed by the holder element 920.

Referring to FIGS. 9A and 9B, the upper and lower halves 920a, 920b of the holder element 920 each include a plurality of side edges 910 and recesses or orifices to accommodate any tubes 60 within the ports 30 of a bio-container 100, 100a, 700a, 800a (see FIGS. 1, 2, 7A, 8E) installed within the element 920. It should be understood, however, that the holder element 920 and its halves 920a, 920b are depicted in an exemplary rectangular configuration in FIGS. 9A and 9B, but can be configured with many other shapes, largely dependent on the shape of the bio-container to be enclosed within the holder element 920. For example, a bio-container with a cylindrical shape factor can be enclosed by similarly shaped holder element 920 and halves 920a, 920b with a cylindrical shape factor. Still further, it is conceivable that the holder element 920, and its halves 920a, 920b, are configured in a size and shape to hold multiple bio-containers. For example, a plurality of cubic-shaped bio-containers can be enclosed by a rectangular-shaped holder element 920.

The holder element 920 depicted in FIGS. 9A and 9B also includes a cushion 950a, 950b, as coupled, or otherwise affixed, to the respective holder element upper and lower halves 920a, 920b. In some embodiments, the cushions 950a, 950b are located in recesses within the halves 920a, 920b. These cushions 950a, 950b are configured to provide cushioning and support to peripheral surfaces 660a of the bio-container 100, 100a, 700a, 800a (see FIGS. 1, 2, 7A, 8E). A suitable polymeric and/or elastomeric cushioning material can be employed for the cushion 950a, 950b, preferably an elastomeric material such as a rubber, vinyl, synthetic rubber and others. It should also be understood that the cushion employed in the holder element 920 can be configured on one or both of the halves 920a, 920b, depending on the configuration and properties of the bio-container 100, 100a, 700a, 800a.

According to certain embodiments of the holder element 920 depicted in FIGS. 9A and 9B, the cushion 950a, 950b can also be configured to protect and contact the edges of the bio-container 100, 100a, 700a, 800a. Edge cushioning of the bio-container, for example, is advantageous for bio-containers that are fabricated without compressive stress states at their edges, e.g., as may be the case when a trimming procedure, such as the trimming operation 400 (see FIG. 6), is conducted and excess glass at the edges of the bio-container is removed after formation. Further, according to an additional embodiment, a polymeric layer (not shown) can be formed or otherwise wrapped over the bio-container 100, 100a, 700a, 800a prior to installation in the holder element 920 to afford the glass portion of the container with additional protection, particularly impact resistance and to mitigate against the development of flaws in the glass of the bio-container. In addition, such a polymeric layer can also provide a safety benefit for the bio-container 100, 100a, 700a, 800a in the unlikely event of its fracture or breakage. More particularly, the polymeric layer can ensure that the contents of the bio-container 100, 100a, 700a, 800a do not leak and that any fractured pieces of the bio-container are otherwise contained within the layer. The polymeric layer of these embodiments can be fabricated from various polymeric materials, including but not limited to, polyvinyl butyral (PVB), polyethylene (PE) and other polymeric materials suitable for molding and wrapping over a glass element.

Referring again to FIGS. 9A-9D, certain aspects of the holder element 920 can include a handle 970, configured for transport and handling of the bio-container assembly 900 containing the bio-container 100, 100a, 700a, 800a (see FIGS. 1, 2, 7A, 8E) and contents within it. The handle 970 can take on any of a variety of shapes and configurations suitable for this function. Further, it may be coupled to one or both of the holder element halves 920a, 920b.

According to another embodiment, the holder element 920 depicted in FIGS. 9A-9D can be configured with a plurality of ribs 995 (see FIG. 9C). The ribs 995 can be located on one or more side edges 910 of the upper and/or lower halves 920a, 920b of the holder element 920. More particularly, the ribs 995 are protrusions, set-offs or other raised features that function to aid in standing the holder element 920 on one of its side edges 910 for purposes of storage and transport. For example, the ribs 995 can aid in allowing the bio-container assembly 900 to stand on a substantially flat surface. Another benefit of the ribs is that they minimize the surface area of the flat surface in contact with the holder element 920, thus reducing the likelihood of contamination.

According to a further embodiment, the holder element 920 depicted in FIGS. 9A-9D can be configured with a plurality of feet 990 (see FIGS. 9B and 9D). The feet 990 can be located on one or both of the upper and lower halves 920a, 920b of the holder element 920. More particularly, the feet 990 are raised features that function to aid in allowing the holder element 920 to stand on one of its primary surfaces, e.g. on a substantially flat surface, for purposes of storage and transport. Another benefit of the feet 990 is that they minimize the surface area of the flat surface in contact with the holder element 920, thus reducing the likelihood of contamination. In addition, the feet 990 can offer an additional stacking capability as they can be configured to fit within recesses 991 of one or more upper and lower holder element halves 920a, 920b (see FIG. 9D).

In another implementation of the holder element 920, as depicted in FIGS. 9A-9D, a plurality of tabs 980 (see FIG. 9B) can be affixed to one or both of the holder element halves 920a, 920b. These tabs 980 can take on a variety of configurations, and function to secure the holder halves 920a, 920b together over the bio-container 100, 100a, 700a, 800a (see FIGS. 1, 2, 7A, 8E). In certain aspects, the tabs 980 are configured to secure the halves 920a, 920b over the bio-container as a complete holder element 920 such that the halves 920a, 920b cannot be readily removed. Such a configuration can be beneficial to ensure that a bio-container assembly 900 with such a holder element 920 is employed in a single-use capacity, if desired. In other aspects, the tabs 980 are configured to fasten the holder halves 920a, 920b together over the bio-container such that they can be removed after fastening, as desired. Such a configuration can be beneficial to allow the holder element 920 to be removed from the bio-container if a multi-use capability is desired. Further, a holder element 920 that can house multiple bio-containers could benefit from tabs 980 configured to allow for removal or separation of the halves 920a, 920b, as this capability can facilitate the use or re-arrangement of one or more bio-containers housed in the element 920.

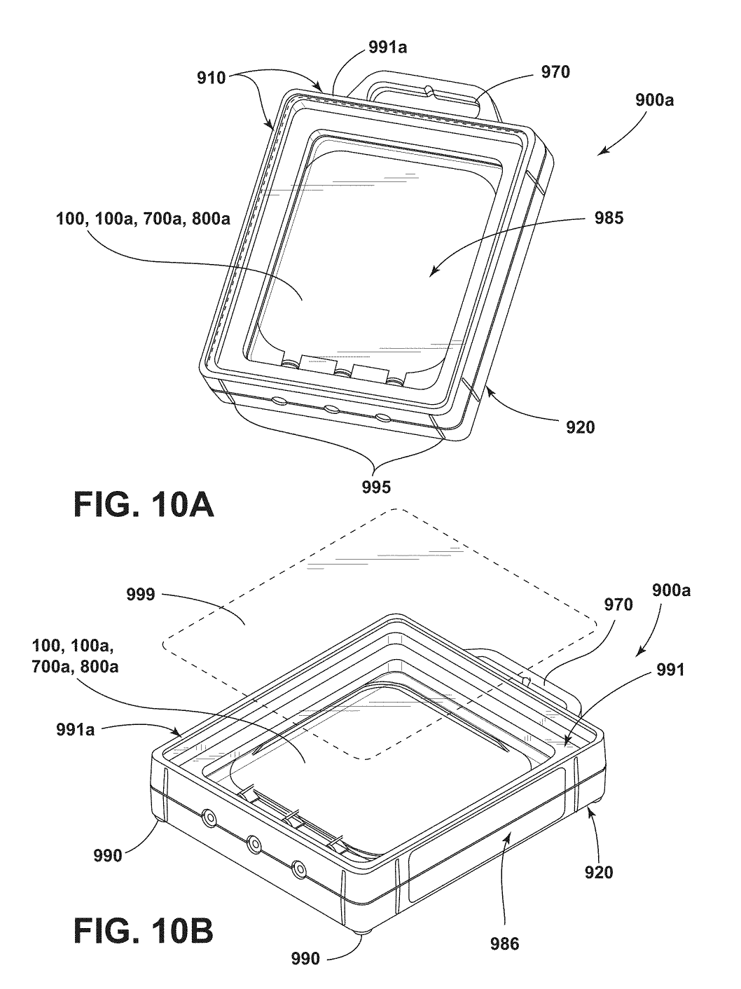

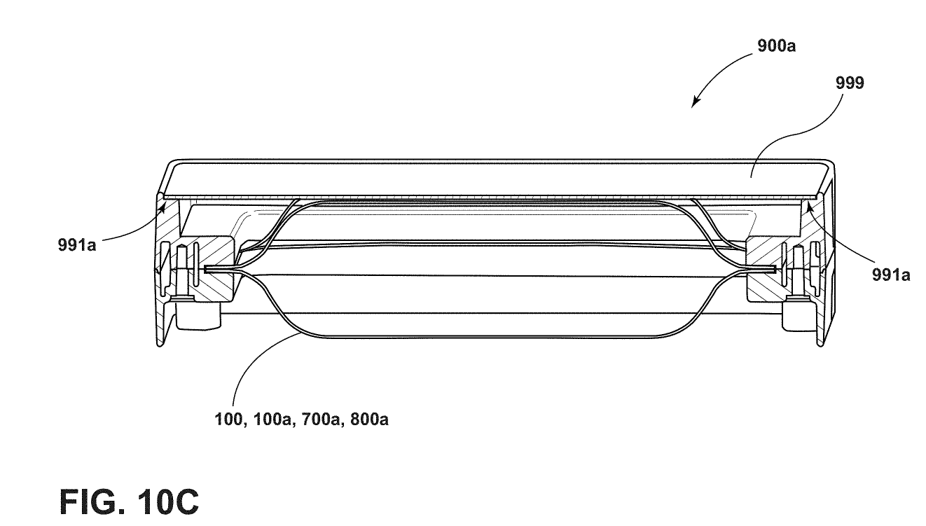

According to another embodiment, a bio-container assembly 900a is depicted in FIGS. 10A-10C according to a further aspect of the disclosure. The bio-container assembly 900a is similar in most respects to the bio-container assembly 900 depicted in FIGS. 9A-9D, and like-numbered elements have the same or similar structure and function. More particularly, the bio-container assembly 900a can, in some implementations, include a bio-container 100, 100a, 700a, 800a (see FIGS. 1, 2, 7A, 8E). The bio-container assembly 900a also includes a holder element 920 with upper and lower halves 920a, 920b that comprise a polymeric material. In addition, and unlike the bio-container assembly 900, the bio-container assembly 900a depicted in FIGS. 10A-10C also includes a translucent panel 999, which is sealed (e.g., via ultrasonic welding) or otherwise fitted (e.g., with an adhesive) onto a periphery portion 991a of the holder element 920. As depicted in FIG. 10B, the panel 999 is installed in an upper half 920a (see FIG. 9A) of the holder element 920; however, a similar panel 999 may also be installed on a lower half 920b (see FIG. 9A) of the holder element 920. Various polymeric materials are suitable for the translucent panel 999 including, but not limited to, polystyrene, acrylonitrile butadiene styrene and polycarbonate. According to some embodiments, the panel 999 is fabricated from a polymeric material that offers high ultra-violet (UV) radiation absorption, to further protect the contents of the bio-container 100, 100a, 700a, 800a. Further, the panel 999 may have a thickness that ranges from about 2 mm to about 2 cm, preferably about 5 mm to about 1 cm.

Referring again to FIGS. 10A-10C, the bio-container assembly 900a, like the bio-container assembly 900 (see FIGS. 9A-9D), has all of the functions, benefits and advantages of its bio-containers, as outlined earlier in the disclosure. Further, the addition of the holder element 920 and panel 999 to the bio-container as part of the assembly 900a allows a user to more effectively transport and use the bio-container with less concern over damage to the bio-container and contamination to the contents within it. That is, the holder element 920 in combination with the panel 999 of the bio-container assembly 900a can act as an additional safety and/or barrier to the bio-container. In addition, the rigidity and structural features of the holder element 920 and panel 999 improve the ease of handling and transport of the bio-container and the contents within it, as the bio-container assembly 900 can be stacked and handled with relative ease. Still further, the inclusion of the panel 999 ensures that the bio-container 100, 100a, 700a, 800a cannot be directly impacted by handling, sharp objects and other foreign objects that could otherwise damage it. Further, the inclusion of the panel 999 reduces the potential for contamination of the contents within the bio-container 100, 100a, 700a, 800a as it reduces the possibility of direct human contact with the surfaces of the bio-container. In addition, these benefits of the bio-container assembly 900a are maintained without a significant loss in the ability of a user to view the contents of the bio-container 100, 100a, 700a, 800a through the panel 999, as the panel is preferably translucent.

### EXAMPLES

The following example represents certain non-limiting embodiments of the disclosure.

### Example 1

A container half, e.g., comparable to a container half 20a, 20b, having the following composition was fabricated according to principles of the disclosure: 61.64% SiO₂, about 17.93% Al₂O₃, 8% B₂O₃, 6.89% Na₂O, 5.36% MgO, and 0.18% SnO₂ (by weight). Permitted Daily Exposure (PDE) levels were measured for this container according to the following protocol (i.e., as set forth in the “Guideline for Elemental Impurities,” Draft Consensus Guideline of the International Conference on Harmonisation of Technical Requirements for Registration of Pharmaceuticals for Human Use, Jul. 26, 2013): incubation of the glass container with distilled water at a 1:6 glass container surface area/water ratio at 50° C. for 48 hours. The results are outlined below in Table 1, as obtained through standard inductively coupled plasma mass spectrometry (ICP-MS) analysis techniques. In particular, Table 1 lists the results for the glass container in comparison to PDE levels for each of the specified elements. As demonstrated by Table 1, the leached amounts of each of the elements (e.g., As, Cd, Hg, etc.) are well below the PDE levels.

TABLE 1

| | | Parenteral PDE | Parenteral PDE | Glass container |
|---|---|---|---|---|
| Element Name | Class | (μg/day)* | (in ppm)** | (in ppm)*** |
| As | 1 | 15.0 | 3.0 | <0.010 |
| Cd | 1 | 6.0 | 1.2 | <0.010 |
| Hg | 1 | 4.0 | 0.8 | ND |
| Pb | 1 | 5.0 | 1.0 | <0.010 |
| Co | 2A | 5.0 | 1.0 | <0.010 |
| Mo | 2A | 180.0 | 36.0 | <0.010 |
| Se | 2A | 85.0 | 17.0 | <0.010 |
| V | 2A | 12.0 | 2.4 | <0.010 |
| Ag | 2B | 35.0 | 7.0 | <0.050 |
| Au | 2B | 130.0 | 26.0 | <0.050 |
| Ir | 2B | 10.0 | 2.0 | <0.050 |
| Os | 2B | 10.0 | 2.0 | ND |
| Pd | 2B | 10.0 | 2.0 | <0.050 |
| Pt | 2B | 10.0 | 2.0 | <0.010 |
| Rh | 2B | 10.0 | 2.0 | <0.010 |
| Ru | 2B | 10.0 | 2.0 | <0.010 |
| Tl | 2B | 8.0 | 1.6 | <0.010 |
| Ba | 3 | 1300.0 | 260.0 | <0.005 |
| Cr | 3 | 1100.0 | 220.0 | <0.010 |
| Cu | 3 | 130.0 | 26.0 | <0.010 |
| Li | 3 | 390.0 | 78.0 | <0.100 |
| Ni | 3 | 60.0 | 12.0 | <0.010 |
| Sb | 3 | 600.0 | 120.0 | <0.010 |
| Sn | 3 | 640.0 | 128.0 | <0.010 |

*PDE values are from Table A.2.1 of “Guideline for Elemental Impurities,” Draft Consensus Guideline of the International Conference on Harmonisation of Technical Requirements for Registration of Pharmaceuticals for Human Use, Jul. 26, 2013.
**ppm was calculated based on 5 mL daily dose
***Determined by ICP-MS

Many variations and modifications may be made to the above-described embodiments of the disclosure without departing substantially from the spirit and various principles of the disclosure. All such modifications and variations are intended to be included herein within the scope of this disclosure and protected by the following claims.

What is claimed is:

1. A holder for a bio-container, comprising:
a holder element comprising a polymeric material and a window; and
a cushion affixed to the holder element in contact with one or more primary exterior surfaces of the bio-container,
wherein the element, cushion and the window are configured to enclose the bio-container, the bio-container viewable through the window.

2. The holder of claim 1, further comprising:
a handle affixed to the holder element.

3. The holder of claim 1, wherein the cushion is further configured to be in contact with side edges of the bio-container.

4. The holder of claim 1, further comprising:
a plurality of ribs affixed to the holder element that are located and sized such that a side edge of the holder and the bio-container can rest on a substantially flat surface.

5. The holder of claim 1, further comprising:
a plurality of feet affixed to the holder element that are located and sized such that the holder element can be stacked onto another holder element and a primary side of the holder element can rest on a substantially flat surface.

6. The holder of claim 1, further comprising:
a plurality of tabs affixed to the holder element, the tabs configured to secure the holder over the bio-container.

7. The holder of claim 1, further comprising:
a plurality of tabs affixed to the holder element, the tabs configured to removably fasten the holder over the bio-container.

8. A holder for a bio-container, comprising:
an upper and a lower holder element, each element comprising a polymeric material;
an upper cushion affixed to the upper element in contact with an upper peripheral exterior surface of the bio-container; and
a lower cushion affixed to the lower element in contact with a lower peripheral exterior surface of the bio-container,
wherein the upper and lower elements and cushions are configured to enclose the bio-container.

9. The holder of claim 8, further comprising:
a handle affixed to at least one of the holder elements.

10. The holder of claim 8, wherein the upper and lower cushions are further configured to be in contact with side edges of the bio-container.

11. The holder of claim 8, further comprising:
a plurality of ribs affixed to at least one of the holder elements that are located and sized such that a side edge of the holder and the bio-container can rest on a substantially flat surface.

12. The holder of claim 8, further comprising:
a plurality of feet affixed to the lower holder element that are located and sized such that the holder element can be stacked onto another holder element and a primary side of the holder element can rest on a substantially flat surface.

13. The holder of claim 8, further comprising:
a plurality of tabs affixed to at least one of the holder elements, the tabs configured to secure the holder over the bio-container.

14. The holder of claim 8, further comprising:
a plurality of tabs affixed to at least one of the holder elements, the tabs configured to removably fasten the holder over the bio-container.

15. A bio-container assembly, comprising:
a bio-container having an interior surface, an exterior surface, and a container thickness from about 0.2 mm to about 2 mm;
at least one port coupled to the bio-container;
a holder element comprising a polymeric material; and
a cushion affixed to the holder element in contact with one or more primary exterior surfaces of the bio-container,
wherein the element and the cushion are configured to enclose the bio-container, and
further wherein the bio-container has a glass composition comprising no materials that are leachable in excess of a Permitted Daily Exposure (PDE) upon exposure to contents of the bio-container.

16. The bio-container assembly according to claim 15, wherein the bio-container further comprises a glass layer spanning the container thickness.

17. The bio-container assembly according to claim 15, wherein the bio-container comprises a compressive stress region that extends to a selected depth in the thickness and a maximum compressive stress at one or both of the interior and exterior surfaces.

18. The bio-container assembly according to claim 17, wherein the compressive stress region comprises a plurality of ion-exchangeable ions and a plurality of ion-exchanged ions.

19. The bio-container assembly according to claim 17, wherein the container further comprises a laminated sheet having a plurality of glass layers spanning the bag thickness.

20. The bio-container assembly according to claim 19, wherein the plurality of glass layers comprise glass compositions with a coefficient of thermal expansion (CTE) mismatch and the compressive stress region is based at least in part on the CTE mismatch.

21. The bio-container assembly according to claim 19, wherein the plurality of glass layers comprise a core layer, outer clad layer and an inner clad layer, the clad layers having a lower coefficient of thermal expansion (CTE) than the core layer and each of the layers having a softening point within 200° C. of the softening point of the other layers.

22. The bio-container assembly according to claim 15, wherein the bio-container comprises a first half, a second half, and a seam that joins the halves.

23. The bio-container assembly according to claim 22, wherein the bio-container further comprises an interior volume from about 1 L to about 200 L.
